(19) United States
(12) Patent Application Publication
(10) Pub. No.: US 2022/0325339 A1
(43) Pub. Date: Oct. 13, 2022

(54) NUCLEIC ACID ANALYSIS

(71) Applicant: Agency for Science, Technology and Research, Singapore (SG)

(72) Inventor: Wee Siong GOH, Singapore (SG)

(21) Appl. No.: 17/612,952

(22) PCT Filed: May 21, 2020
(86) PCT No.: PCT/SG2020/050301

§ 371 Date: Nov. 19, 2021

(30) Foreign Application Priority Data
May 21, 2019 (SG) 10201904542W

(51) Int. Cl. C12Q 1/6869; C12Q 1/6804; G01N 33/53

(52) U.S. Cl. CPC C12Q 1/6869; C12Q 1/6804; G01N 33/5308; C12Q 2600/154

(57) ABSTRACT

The present invention relates to a method for analysis of methylation of ribonucleic acid (RNA) comprising the steps: (i) contacting RNA with one or more antibodies which binds to methylated site(s) of RNA; wherein the methylated site(s) comprise at least one ribonucleotide base modified by one or more methyl groups; (ii) photo-crosslinking the one or more antibodies to crosslink individual antibodies to the RNA molecule(s) to form RNA-antibody conjugates; (iii) immunoprecipitating to separate the RNA-antibody conjugates; (iv) treating the RNA-antibody conjugates with at least one exonuclease; (v) removing the crosslinked antibodies from the RNA-antibody conjugates to release RNA; and (vi) analysing the released RNA.

### FIELD OF THE INVENTION

The present invention relates to the field of nucleic acid analysis, in particular, transcriptome analysis. More in particular, the present invention relates to analysis of methylation of ribonucleic acid.

### BACKGROUND OF THE INVENTION

Out of more than 100 known RNA modifications, N6-methyladenosine (m6A) is the most abundant mRNA modification. The field of m6A gained prominence after next-generation sequencing mapped m6A transcriptome-wide within mRNAs (Dominissini et al., 2012; Meyer et al., 2012). m6A regulates multiple forms of downstream RNA metabolic pathways including but not limited to mRNA decay, mRNA translation, pre-mRNA splicing and pri-miRNA processing (Alarcon et al., 2015a; 2015b; Meyer et al., 2015; Xiao Wang et al., 2014; 2015).

Another prominent RNA modification is N6,2′-O-dimethyladenosine (m6Am), which is formed by the base methylation of 2′-O-methyladenosine (Am) located at the first nucleotide of mRNAs, adjacent to the mRNA cap (C. Wei et al., 1975). m6Am was reported to stabilize mRNA by conferring resistance to mRNA-decapping and might also regulate other forms of RNA metabolism (Mauer et al., 2017).

m6A methylases have been identified in the form of catalytic METTL3 in complex with METTL14, WTAP, KIAA1429 and RBM15/RBM15B (Bokar et al., 1994; 1997; J. Liu et al., 2013; Patil et al., 2016; Ping et al., 2014; Schwartz et al., 2014; Xiang Wang et al., 2016). The METTL3 methylase complex deposits m6A in the ‘RRm6ACH’ (R=A/G, H=A/C/U) motif (Dominissini et al., 2012). However, only a minor fraction of RRACH motifs are methylated, with m6A preferentially localized within the coding DNA sequence (CDS) and 3′ untranslated region (3′UTR) region proximal to the stop codon, and to a lesser extent within the 5′UTR. Mettl3 lesion results in a drastic loss of cellular m6A level, which affects normal cellular differentiation (Batista et al., 2014; Geula et al., 2015). METTL16 is another m6A methylase, which methylates the ‘UACm6AGAGAA’) motif (Pendleton et al., 2017). METTL16 depletion also reduces methylation in regions lacking ‘UACAGAGAA’ motifs, suggesting that METTL16 mediates methylation of non-‘UACAGAGAA’ motifs either directly or indirectly. Additionally, the discovery of both FTO and ALKBH5 as m6A demethylases has led to the proposal that m6A is a dynamic and reversible RNA modification (Jia et al., 2011; Zhao et al., 2018; Zheng et al., 2013). Despite their importance to cellular processes, there have recently been multiple reports with conflicting views on the biological function of ALKBH5 and FTO (Darnell et al., 2018; Ke et al., 2017; Mauer et al., 2017; 2019; Rosa-Mercado et al., 2017; J. Wei et al., 2018; Zhao et al., 2018).

When studying m6A, m6A-RNA-immunoprecpitation-sequencing (m6A-RIP-seq) is the most common m6A-sequencing method utilized but it suffers from poor resolution (˜150 nt). Single base-resolution m6A-sequencing techniques have been developed and these involve crosslinking and immunoprecipitating (CLIP) m6A with specific antibodies to induce truncations or mutations at m6A sites during reverse transcription (Ke et al., 2015; Linder et al., 2015). These m6A-sequencing techniques have revealed new insights into m6A and m6Am regulation of cellular processes, thereby highlighting the benefits and importance of single-base-resolution m6A sequencing (Ke et al., 2017; Mauer et al., 2017; Meyer et al., 2015; Patil et al., 2016). However, these single-base-resolution techniques are generally time-consuming and involve inconvenient procedures such as radioactive gel electrophoresis. Furthermore, they do not include the use of methylated spike in controls to correct for antibody immunoprecipitation efficiency, or a RNA input library prepared in parallel for normalization. Consequently, these techniques are not suitable for quantifying differential methylation between different sample types. This might explain why to date, no effort has been made to precisely map the methylomes specifically mediated by every individual known methylase and demethylase.

It is therefore desirable to develop new and improved new methods to analyse ribonucleic acid methylation.

### SUMMARY OF THE INVENTION

The present invention relates to a method for analysis of methylation of ribonucleic acid (RNA) comprising the steps:

(i) contacting RNA with one or more antibodies which binds to methylated site(s) of RNA; wherein the methylated site(s) comprise at least one ribonucleotide base modified by one or more methyl groups;

(ii) photo-crosslinking the one or more antibodies to crosslink individual antibodies to the RNA molecule(s) to form RNA-antibody conjugates;

(iii) immunoprecipitating to separate the RNA-antibody conjugates;

(iv) treating the RNA-antibody conjugates with at least one exonuclease;

(v) removing the crosslinked antibodies from the RNA-antibody conjugates to release RNA; and

(vi) analysing the released RNA.

It will be appreciated that analysing the RNA includes identifying methylated sites.

### BRIEF DESCRIPTION OF THE FIGURES

FIG. 1. m6ACE treatment enriches for RNA fragments with m6A in the first nucleotide. (See also FIG. 8 and Table 1).

(a) Procedure for m6ACE-seq.

(b-d) m6ACE (green) and Input (orange) read-start counts (in reads per million mapped or RPM) mapped to either a synthetic RNA spike-in sequence (Table 1) with a single m6A at position 21 (B), 18S rRNA (C) or 28S rRNA (D). Known m6A sites are denoted by black dots.

Sequence corresponds to the same strand as the m6A site. Blue horizontal bars represent transcript models.

(e) Metagene distribution profile of all significant m6A/m6Am sites in WT cells.

(f) MEME analysis of the sequence context of all significant m6A/m6Am sites in WT cells.

FIG. 2. m6ACE-seq quantitatively maps m6A reductions at individual METTL3-dependent m6A sites. (See also FIG. 9).

(a) Scatterplot of average RML of WT versus Mettl3-KO cells. METTL3-dependent are sites with RML reductions of at least log₂ fold of 2 (p<0.05) in Mettl3-KO compared to WT cells.

(b,e) m6ACE (green) overlaid on Input (orange) read-start RPM counts mapped to a representative gene. METTL3-independent and dependent sites are respectively denoted by a black or red dot. Sequence corresponds to the same strand as the m6A site. Blue horizontal bars represent transcript models, with green and red sections respectively representing the start and stop codons. Magnified views of the 5′UTR and 3′UTR are also displayed for (b).

For (e), percentages represent proportion of WT RNA in a mixture of WT and Mettl3-KO RNA.

(c) Metagene distribution profile of all METTL3-dependent m6A.

(d) MEME analysis of the sequence context of METTL3-dependent m6A.

(f) Plot of normalized RML against percentage of WT RNA in mixtures of WT and Mettl3-KO.

RML of METTL3-dependent sites in each mixture was normalized to the corresponding RML in 100% WT RNA. Averages of triplicate normalized RML are represented with error bars (standard deviation). A linear regression fit of the plot is depicted with its R2 value and p-value. ## and ### respectively denote p<10⁻¹⁴⁵ and p<10⁻³⁰⁷ (1-tailed T-test).

FIG. 3. m6ACE-seq quantitatively maps RML reductions at individual PCIF1-dependent m6Am sites. (See also FIG. 10).

(a) m6ACE (green) overlaid on Input (orange) read-start RPM counts mapped to a representative gene (center). PCIF1-dependent and METTL3-dependent sites are respectively denoted by red angles and red dots. Sequence corresponds to the same strand as the m6A/m6Am sites. Blue horizontal bars represent transcript models. Magnified views of the 5′UTR (left) and 3′UTR (right) are also displayed.

PCIF1-independent sites are sites where average RML reduction from WT to PCIF-KO is not significantly more than 1.5 fold (1-tailed T-test p<0.05).

(b) Scatterplot of average RML of WT versus Pcif1-KO cells. PCIF1-dependent are sites with RML reduction of at least log₂ fold of 2 (p<0.05) in Pcif1-KO compared to WT cells.

(c) Functional annotation enrichment of genes containing PCIF1-dependent m6Am.

(d) Metagene distribution profile of PCIF1-dependent m6Am.

(e) MEME analysis of the sequence context of PCIF1-dependent m6Am.

(f) Venn diagram representing overlap between 5′UTRs containing PCIF1-dependent m6Am with 5′UTRs containing PCIF1-independent sites. p-value of overlap by chance was calculated with a hypergeometric test.

(g) m6ACE (green) overlaid on Input (orange) read-start RPM counts mapped to a representative 5′UTR. PCIF1-dependent and PCIF1-independent sites are respectively denoted by red angles and black dots. Sequence corresponds to the same strand as the m6A/m6Am sites. Blue horizontal bars represent transcript models. PCIF1-independent sites are sites where average RML reduction from WT to PCIF-KO is not significantly more than 1.5 fold (1-tailed T-test p<0.05).

(h) Pie charts representing alignment of all identified m6A/m6Am (top) or PCIF1-dependent m6Am (bottom) with respect to CAGE-seq annotated TSSs (Abugessaisa et al., 2017).

FIG. 4. METTL16 depletion reduces methylation of a plethora of m6A sites beyond its direct UACAGAGAA targets. (See also FIG. 11).

(a) m6ACE (green) overlaid on Input (orange) read-start RPM counts mapped to the Mat2a 3′UTR. Locations of ‘UACAGAGAA’ and similar motifs are denoted by roman numerals.

Sequence corresponds to the same strand as the m6A site. Blue horizontal bars represent transcript models.

(b) Magnified representations of the Mat2a 3′UTR at positions i, ii and iii as denoted in (3A).

(c) Scatterplot of average RML of WT versus Mettl16-KD cells. METTL16-dependent are sites with RML reduction of at least log₂ fold of 2 (p<0.05) in Mettl16-KD compared to WT cells.

(d) Metagene distribution profile of METTL16-dependent m6A/m6Am.

(e) MEME analysis of the sequence context of METTL16-dependent m6A/m6Am.

(f,g) Venn diagram representing overlap between METTL16-dependent with METTL3-dependent (f) or PCIF1-dependent (g) sites. p-value of overlap by chance was calculated with a hypergeometric test.

FIG. 5. ALKBH5 suppresses accumulation of m6A. (See also FIG. 12).

(a) m6ACE (green) overlaid on Input (orange) read-start RPM counts mapped to a representative gene. ALKBH5-regulated sites are denoted by green dots. Sequence corresponds to the same strand as the m6A sites. Blue horizontal bars represent transcript models.

(b) Scatterplot of average RML of WT versus Alkbh5-KO cells. ALKBH5-regulated are sites with RML accumulation of at least log₂ fold of 1 (p<0.05) in Alkbh5-KO compared to WT cells.

(c) Metagene distribution profile of ALKBH5-regulated sites.

(d) MEME analysis of the sequence contexts of ALKBH5-regulated sites.

(e) Venn diagrams representing overlaps between ALKBH5-regulated sites with METTL3-dependent m6A. p-value of overlap by chance was calculated with a hypergeometric test.

(f) Receiver operator characteristic (ROC) curve for how well ALKBH5-regulated sites are at predicting steady-state non-methylated sites in WT cells. Each curve represents ALKBH5-regulated sites as defined by a different minimum log 2FC RML accumulation cutoff (p<0.05) in Alkbh5-KO over WT cells. Area-under-curve (AUC) values for each log 2FC are also provided.

FIG. 6. FTO loss causes m6Am accumulation that disrupts binding of specific snRNA precursors to nuclear export machinery. (See also FIG. 13).

(a) Scatterplot of average RML of WT versus Fto-KO cells. FTO-regulated (‘X’s) are sites with RML accumulation of at least log₂ fold of 1 (p<0.05) in Fto-KO compared to WT cells.

(b,c) m6ACE (green) overlaid on Input (orange) read-start RPM counts mapped to the 5′ ends of U1 snRNA (b) and U4 snRNA (c). FTO-regulated m6Am sites are denoted by green dots. Sequence corresponds to the same strand as the m6A/m6Am sites. Blue horizontal bars represent transcript models.

(d) Box and whisker plots of m6Am/(m6Am+Am) percentages in WT (blue) and Fto-KO (orange) cells as measured by UHPLC-MS/MS. Represented are median and interquartile ranges for 3 biological replicates. # and ## denote p<0.05 and 0.005 respectively (1-tailed Ttest).

Values for WT cells are in the non-quantifiable range.

(e) Box and whisker plots of ratio of sRNA levels (relative to 7SL scRNA) in nucleus over cytoplasm in WT (blue) and Fto-KO (orange) cells. Represented are median and interquartile ranges for 6 biological replicates, normalized to the corresponding median values in WT cells.

n.s. denotes not-significant, * denotes p<0.1 and ** denotes p<0.01 (1-tailed T-test).

(f) Box and whisker plots of ratios of each sRNA level (relative to Gapdh mRNA) pulled down with anti-NCBP2 over anti-IgG from WT (blue) and Fto-KO (orange) lysates. Represented are median and interquartile ranges for at least 3 biological replicates, normalized to the corresponding median values in WT cells. n.s. denotes not-significant, * denotes p<0.1 and ** denotes p<0.01 (1-tailed T-test).

(g) ROC curve for how well FTO-regulated sites are at predicting steady-state nonmethylated sites in WT cells. Each curve represents FTO-regulated sites as defined by a different minimum log 2FC RML accumulation cutoff (p<0.05) in Fto-KO over WT cells. AUC values for each log 2FC are also provided.

FIG. 7. FTO overexpression causes aberrant mRNA methylation-suppression in the nucleus.

(a) Immunofluorescence images of WT versus Fto-KO cells. Anti-FTO i and anti-FTO ii antibodies are respectively Abcam ab126605 and Santa Cruz sc271713. Anti-FTO i is specific for endogenous FTO while anti-FTO ii is not. Scale bar denotes 10 μm.

(b) Immunofluorescence images of C-terminal-3×-FLAG-tagged-WT-FTO in HEK293T. Scale bar denotes 10 μm.

(c,d) m6ACE (green) overlaid on Input (orange) read-start RPM counts mapped to representative mRNAs. Sequence corresponds to the same strand as the m6A site. Blue horizontal bars represent transcript models.

(e) Scatterplot of average RML of WT versus WT-Fto-OE cells. WT-FTO-affected are sites with RML reduction of at least log₂ fold of 2 (Student's t-test p<0.05) in WT-Fto-OE compared to WT cells.

(f) MEME analysis of the sequence contexts of FTO-affected sites.

(g) Model for cytoplasmic RNA methylation-reversal.

(h) Model for disruptive methylation suppression by RNA demethylases. (Left panel) Most methylated RNAs (blue) are not demethylated by demethylases but simply undergo eventual RNA decay. (Middle panel) Selected RNAs (green) that ought to remain unmethylated are acted on almost simultaneously by both methyltransferase and demethylase, resulting in no net accumulation of RNA methylation. (Right panel) In the absence of demethylases, RNA methylation accumulates anomalously, which disrupts regular RNA processing.

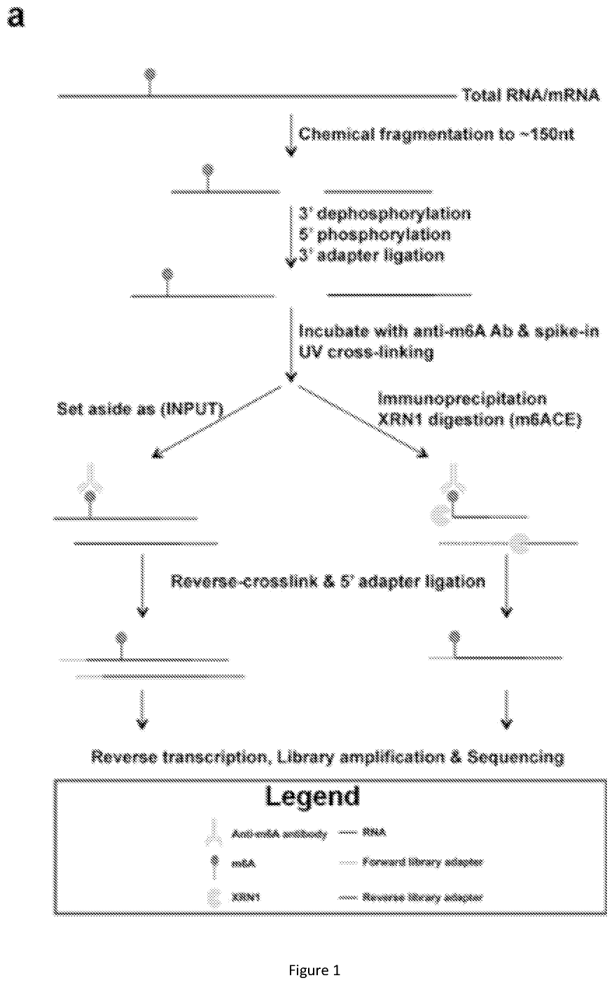

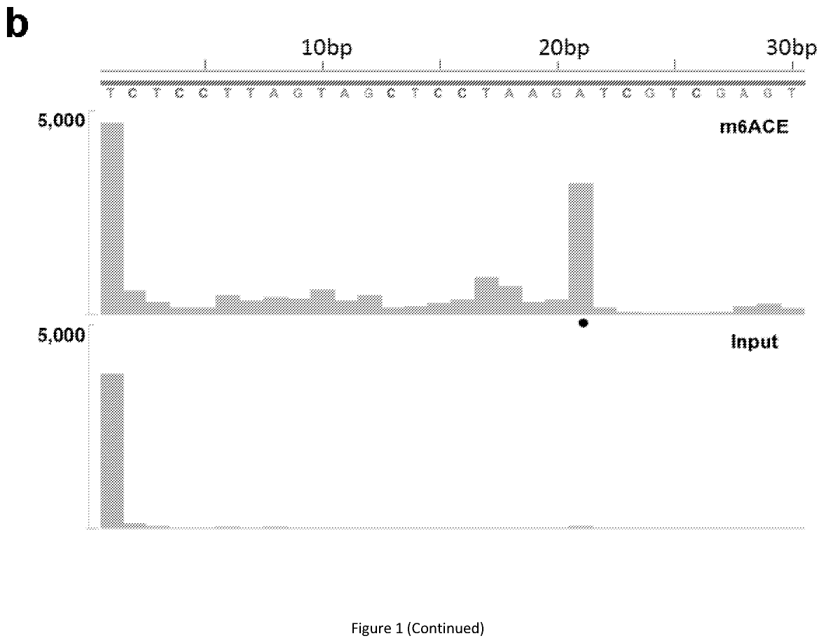

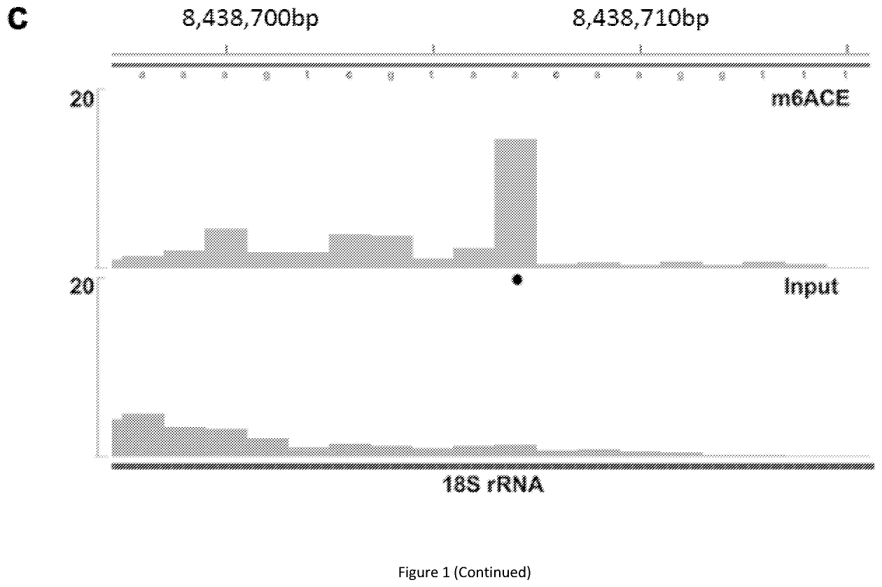

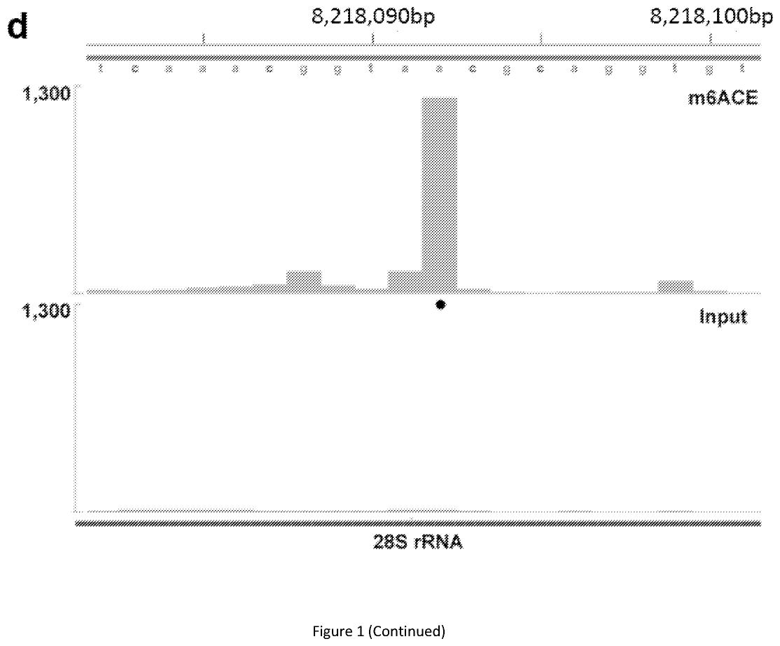

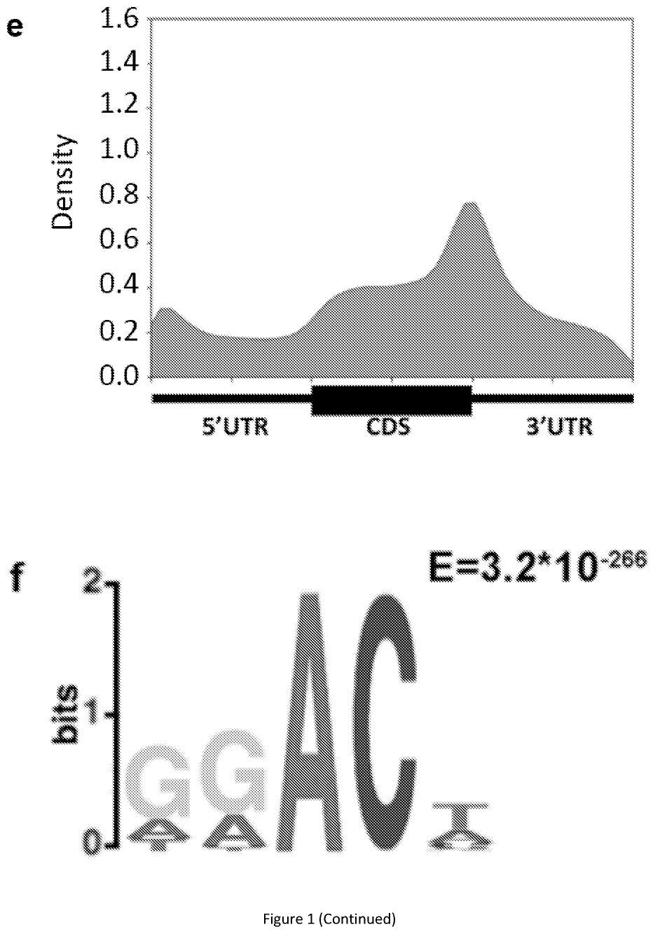

FIG. 8. Validation of m6ACE-seq. (Related to FIG. 1).

(a-d) m6ACE (green) overlaid on Input (orange) read-start RPM counts mapped to representative genes previously validated by SCARLET (N. Liu et al., 2013). Sequence corresponds to the same strand as the m6A site. Blue and black horizontal bars denote SCARLET positive and negative sites respectively.

(e) Overlap in sites identified using m6ACE-seq of HEK293T RNA, miCLIP-seq of HEK293 RNA (Linder et al., 2015) and m6A-CLIP-seq of HeLa cytoplasmic RNA (Ke et al., 2017).

(f) m6ACE (green) overlaid on Input (orange) read-start RPM counts mapped to mitochondrial 16S rRNA. Sequence corresponds to the same strand as the m6A site. Blue horizontal bars represents the 16S rRNA transcript. DNA probes used in T3 DNA ligase assay are also represented.

(g) Schematic outlining T3 DNA ligase assay for m6A detection (W. Liu et al., 2018).

(h) qPCR CT-values as an inverse measure of ligation efficiency of DNA probes depicted in (F). Represented are averages of technical triplicates with standard deviation error bars.

(i) Scatterplot of average RML of a first set of WT triplicate samples versus a second set of WT triplicate samples. R-value and p-value of linear regression fit of the 2 sets of average RMLs are reported.

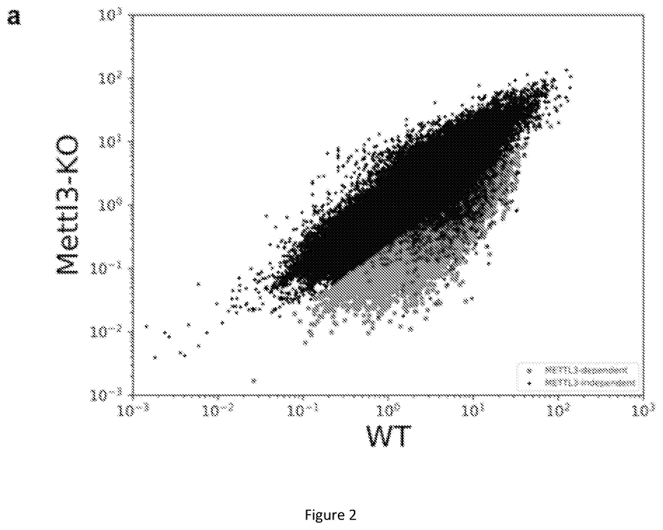

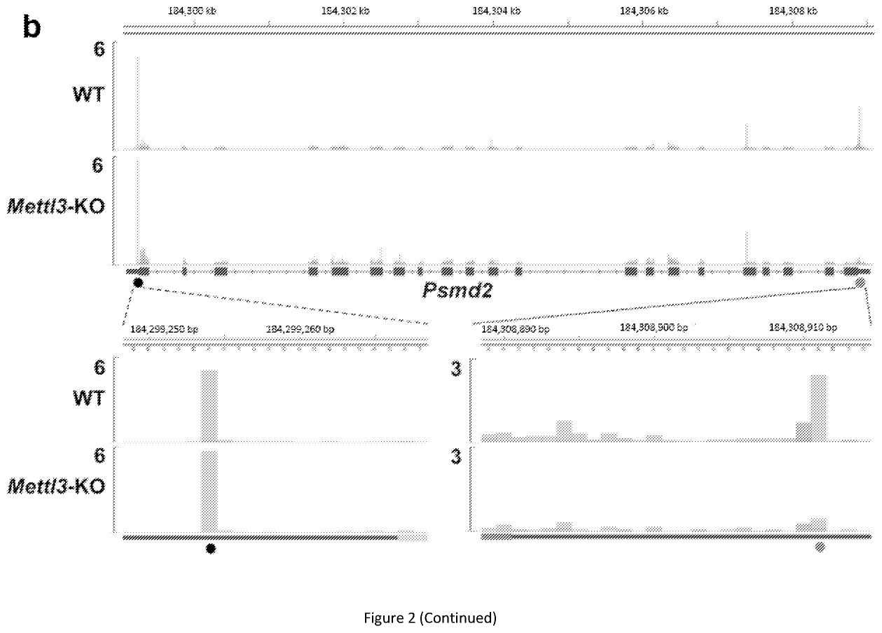

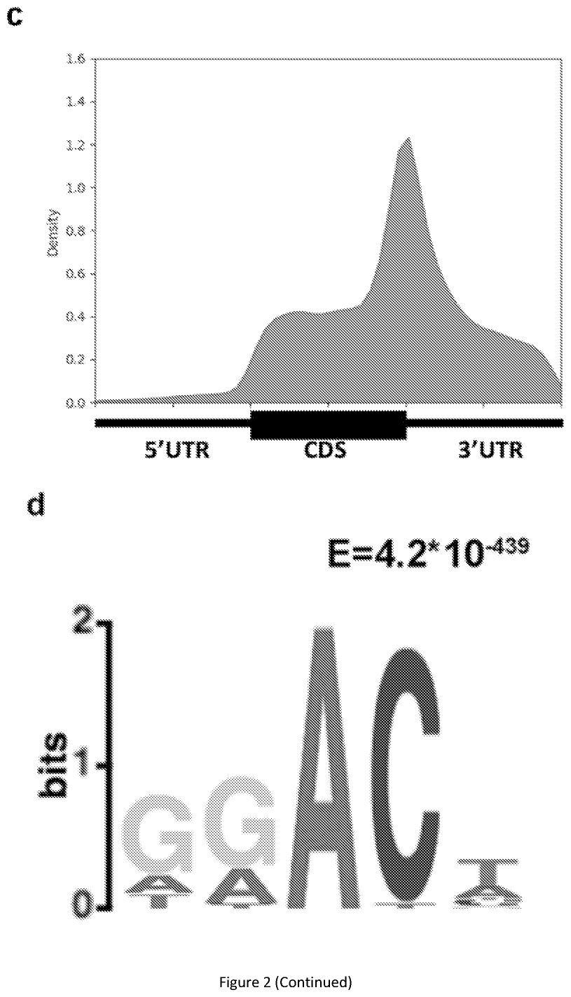

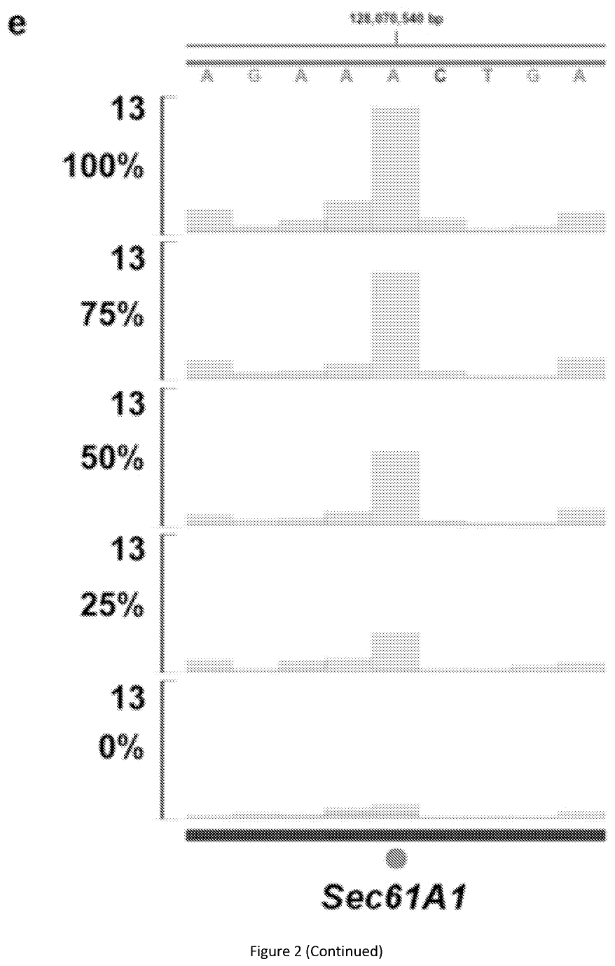

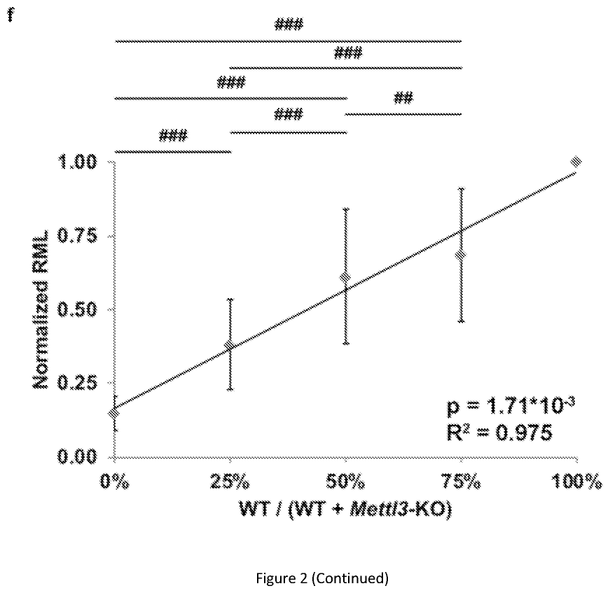

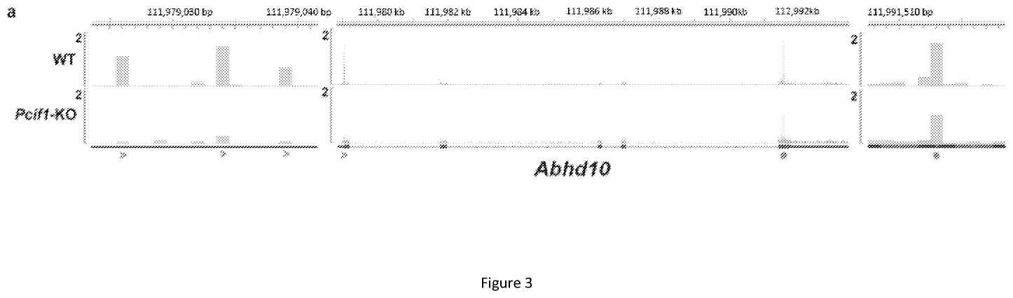

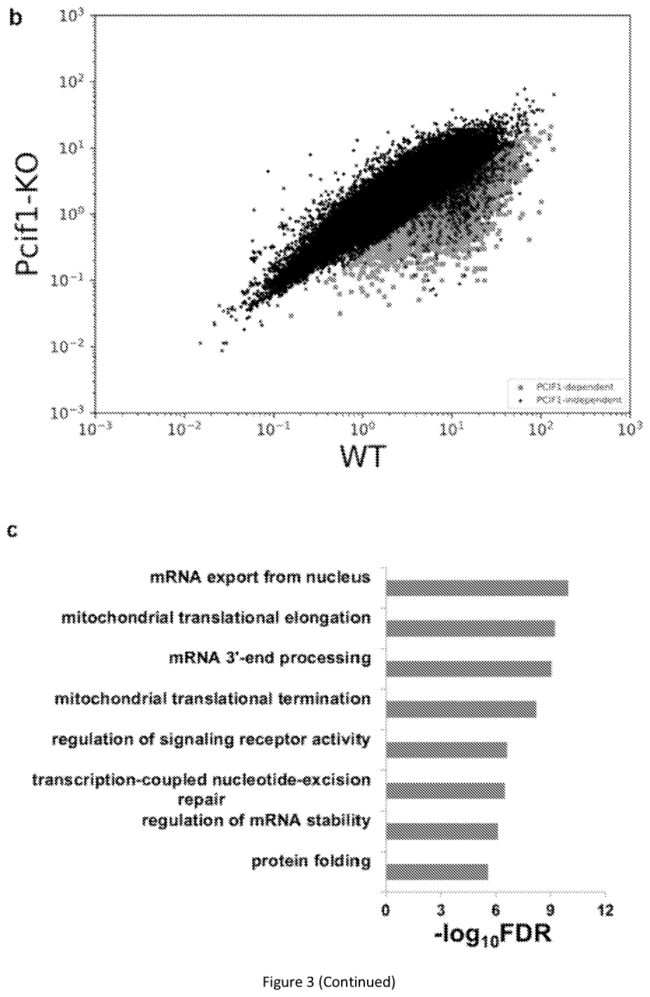

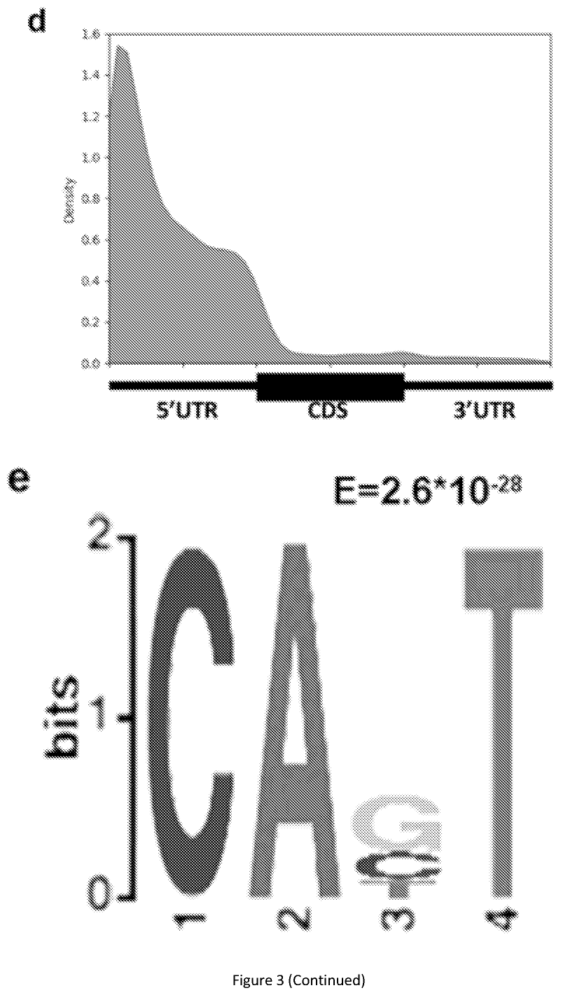

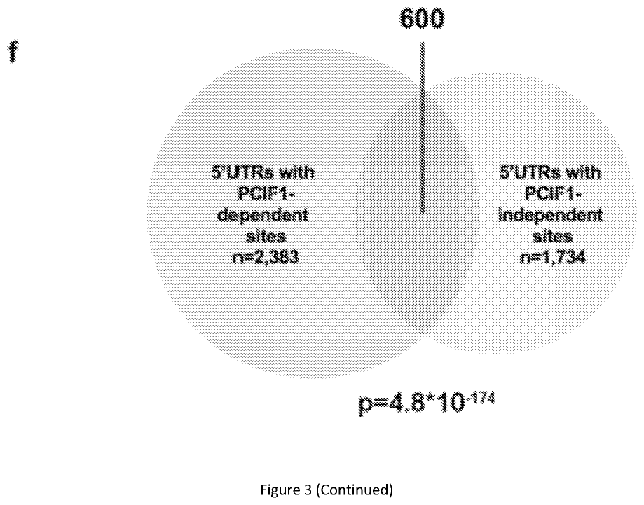

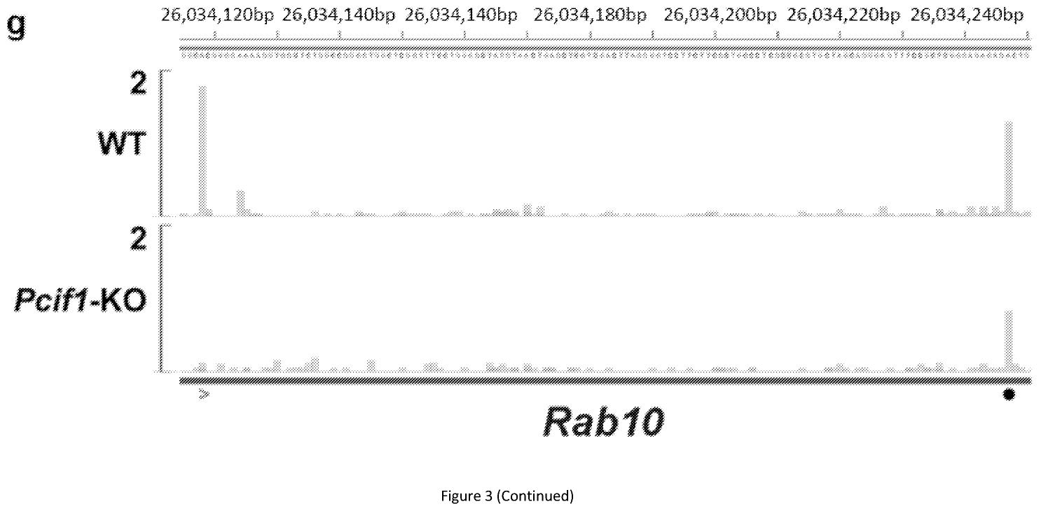

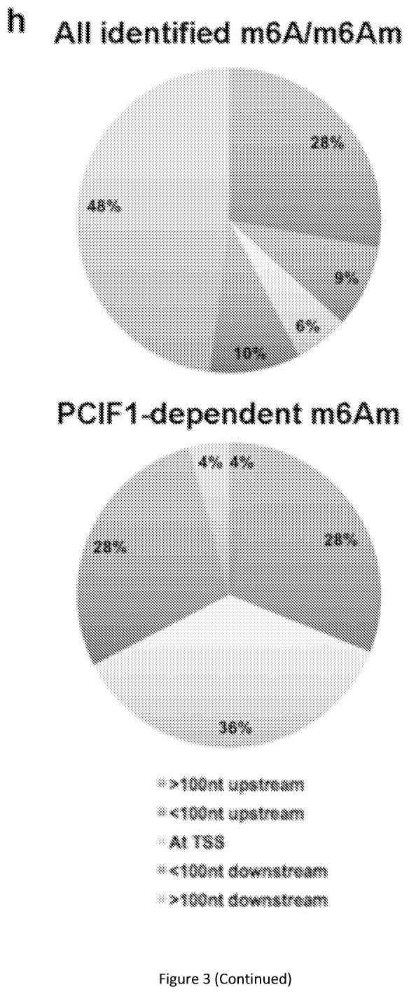

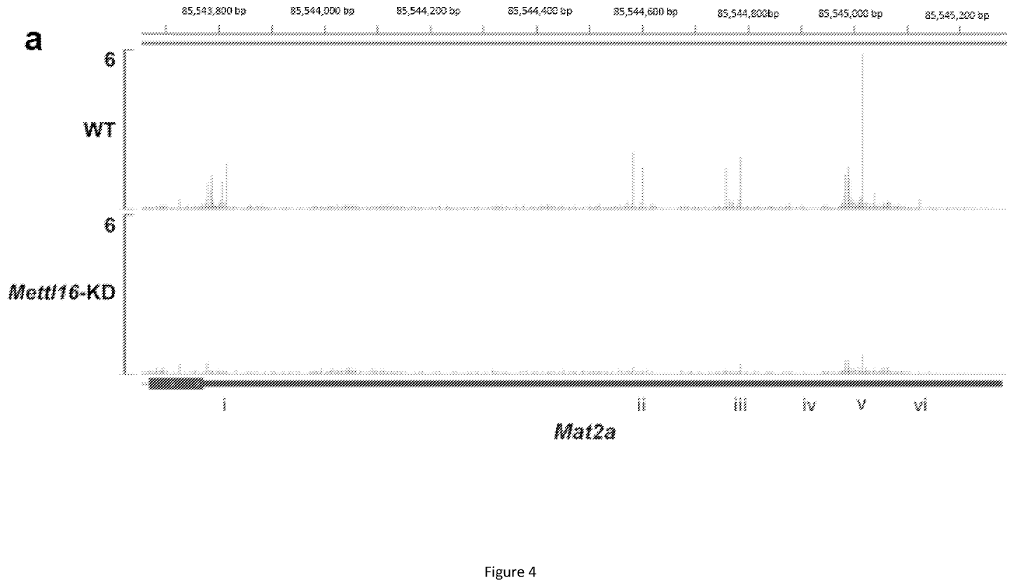

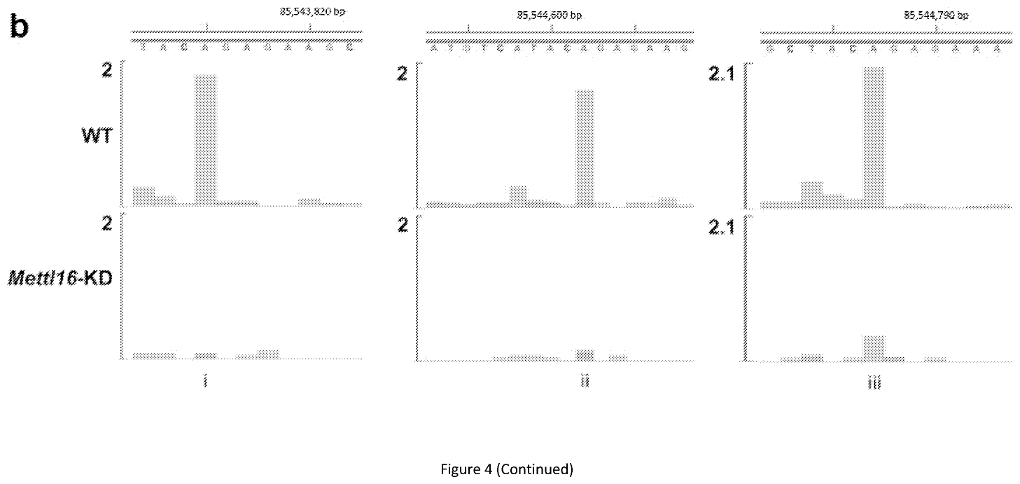

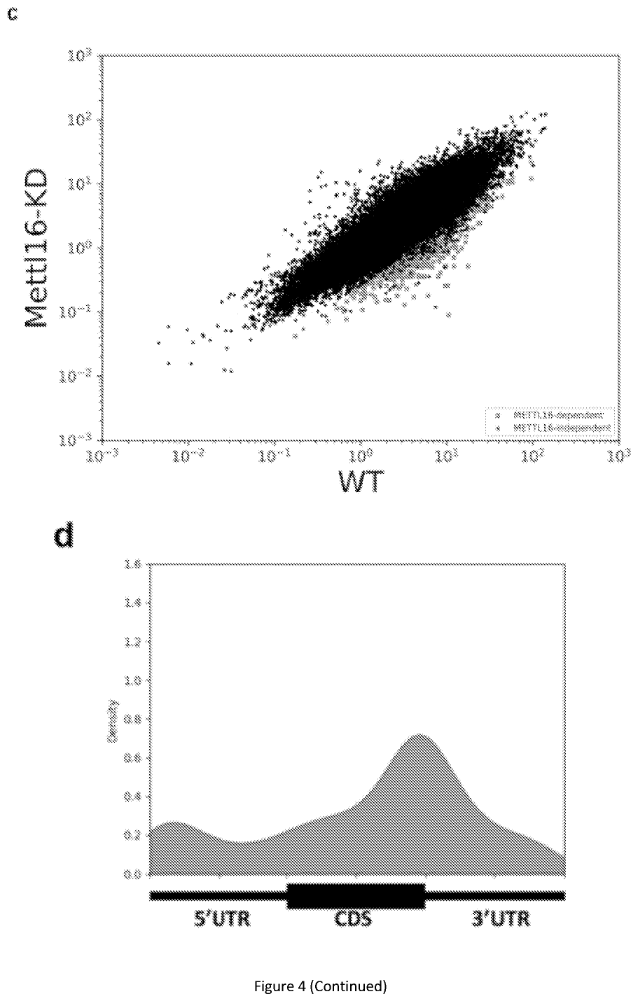

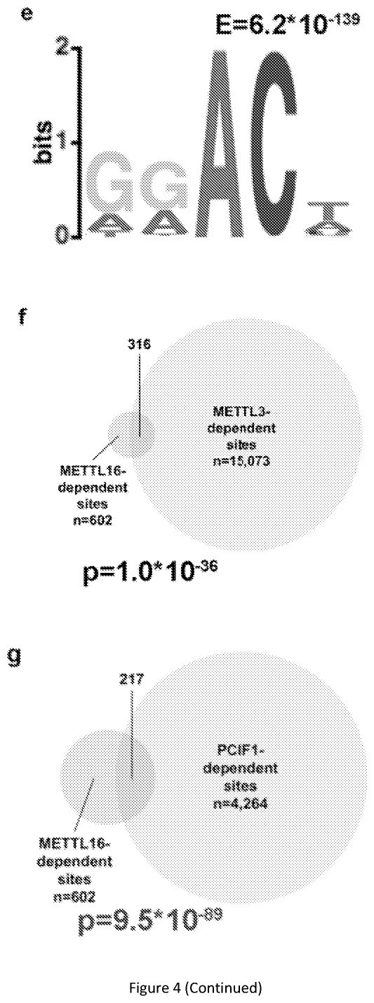

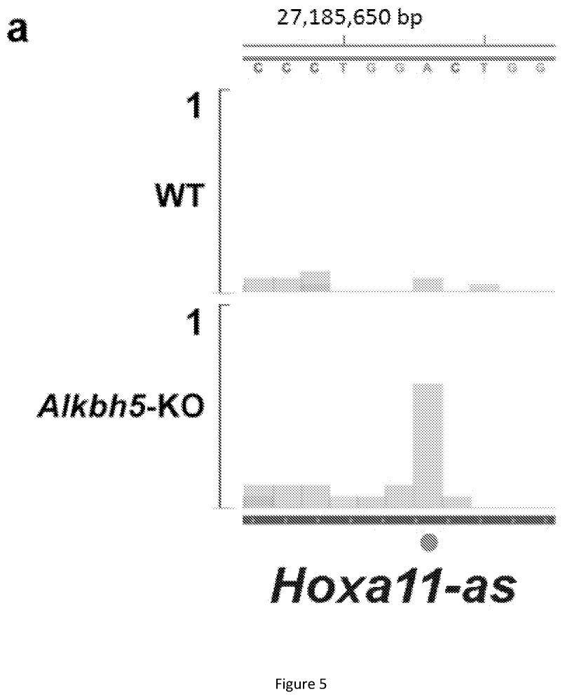

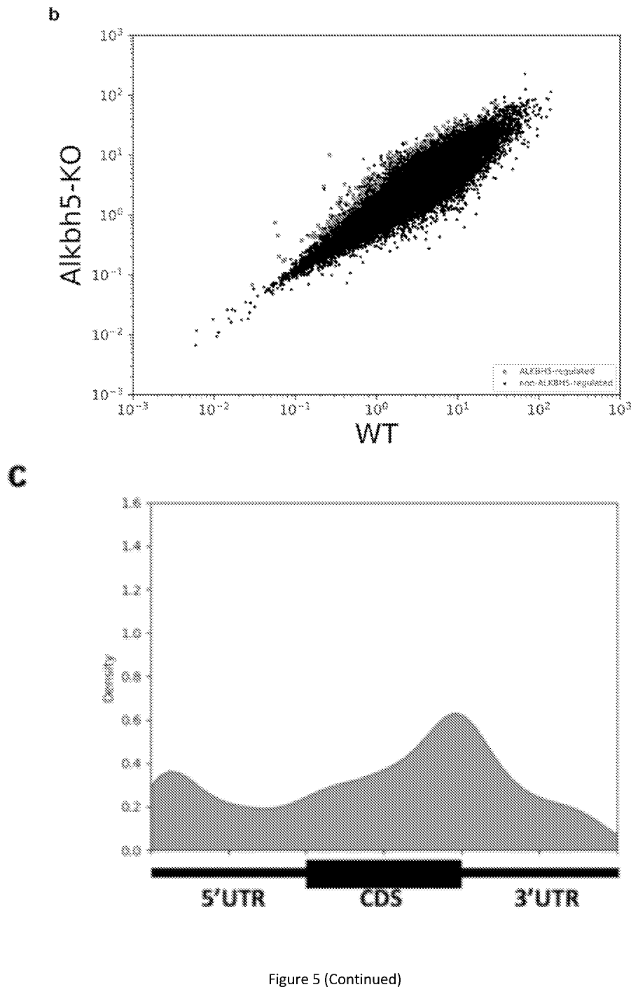

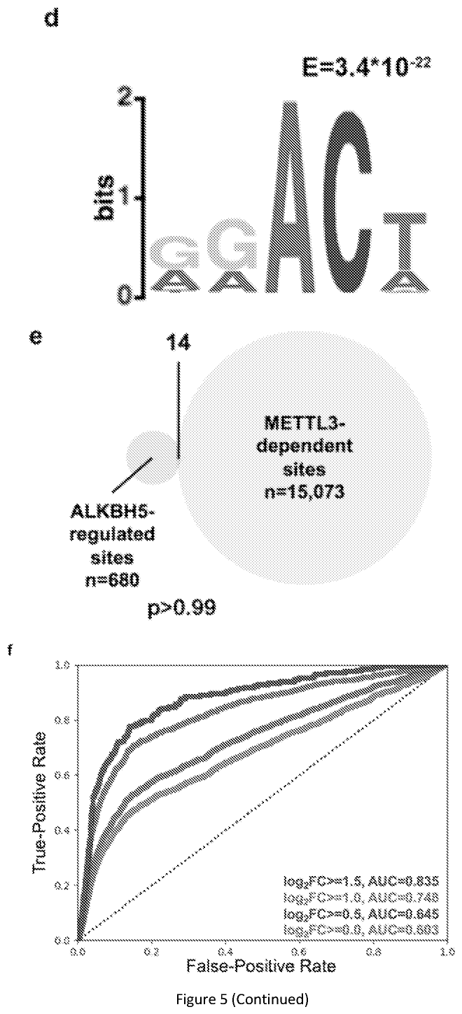

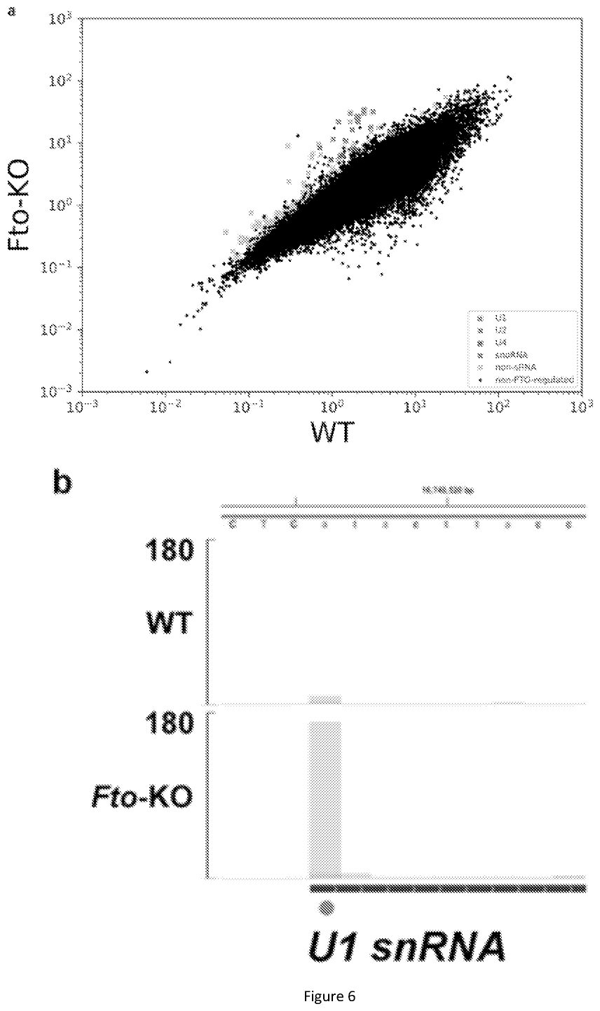

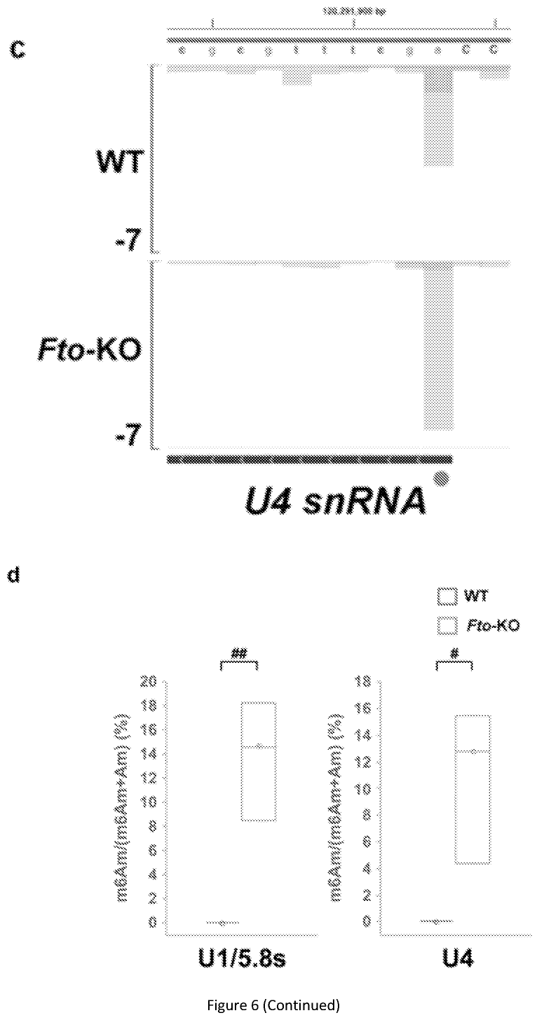

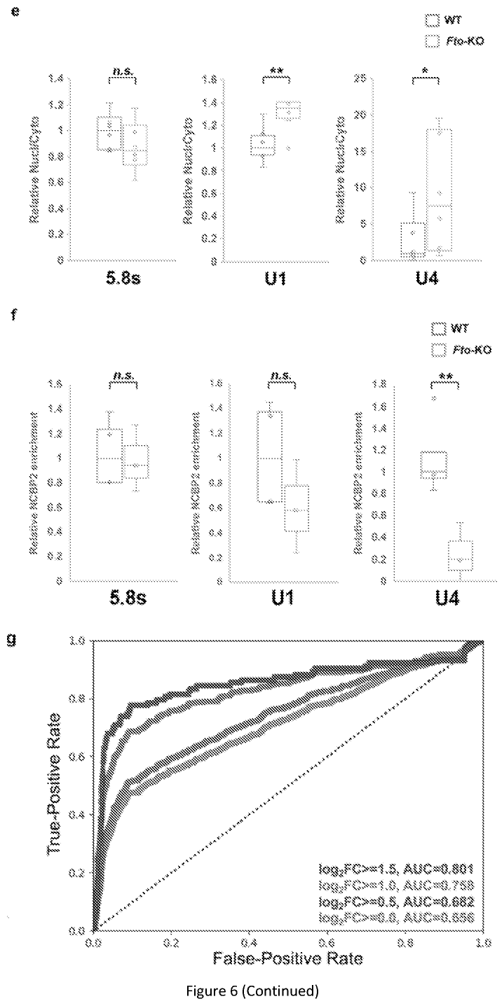

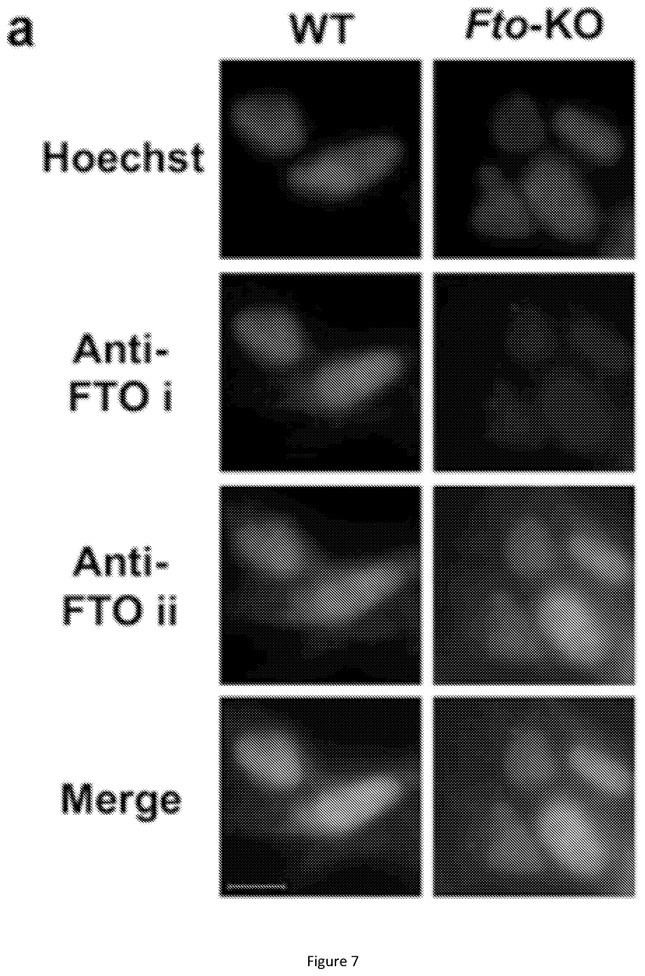

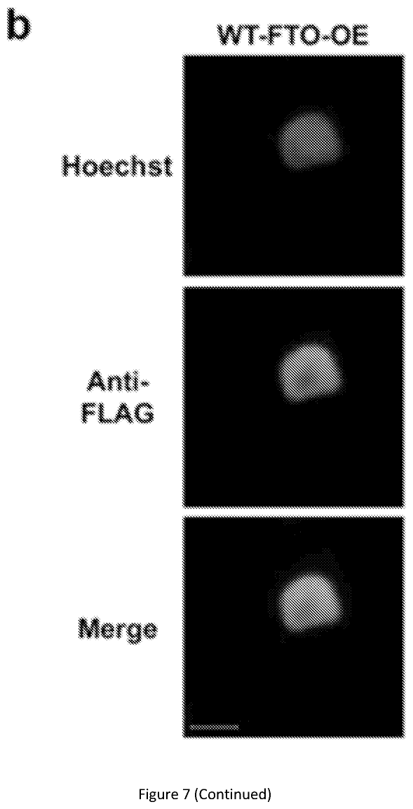

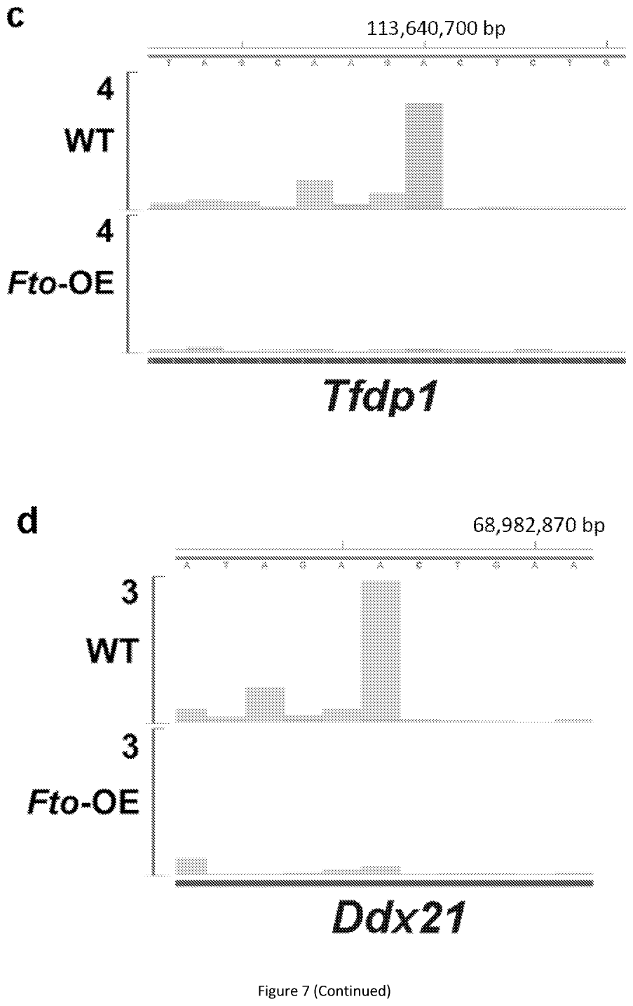

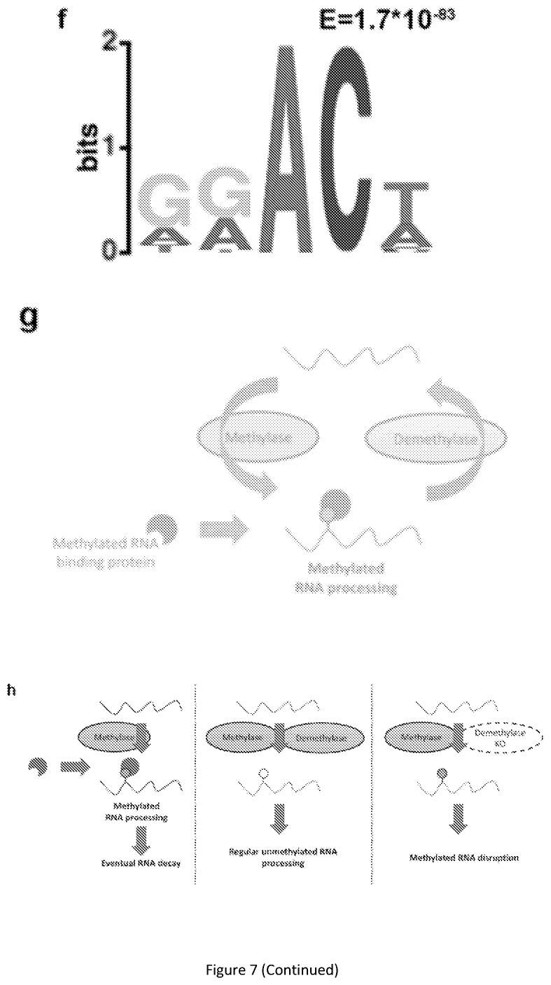

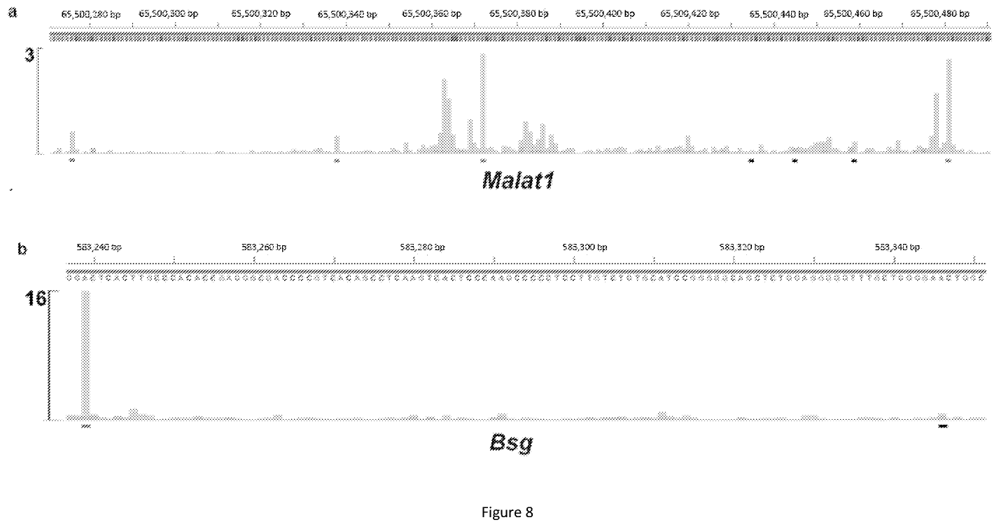

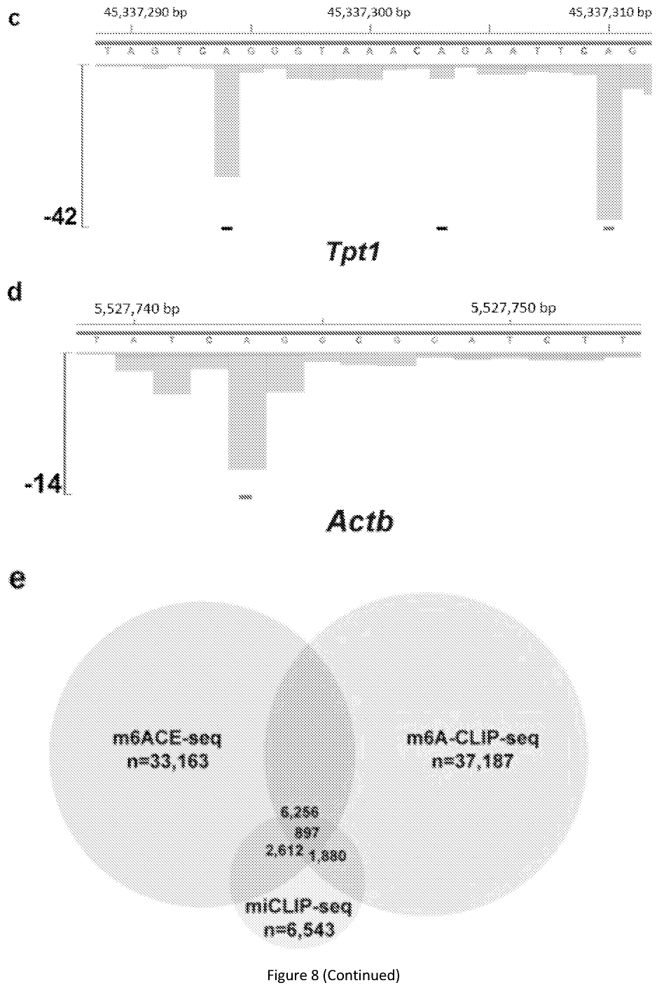

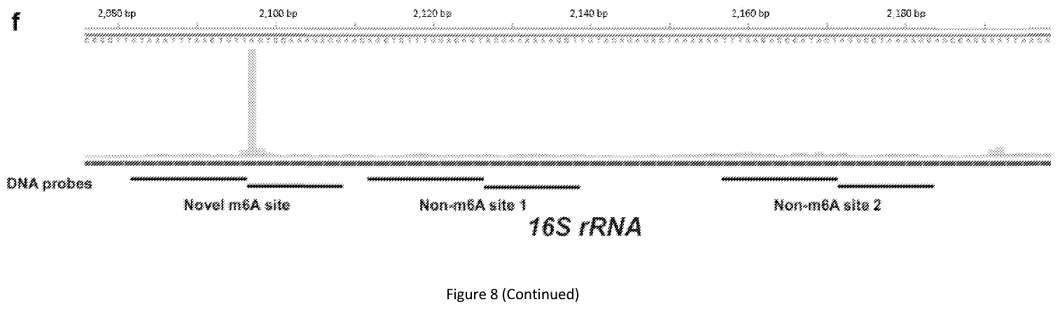

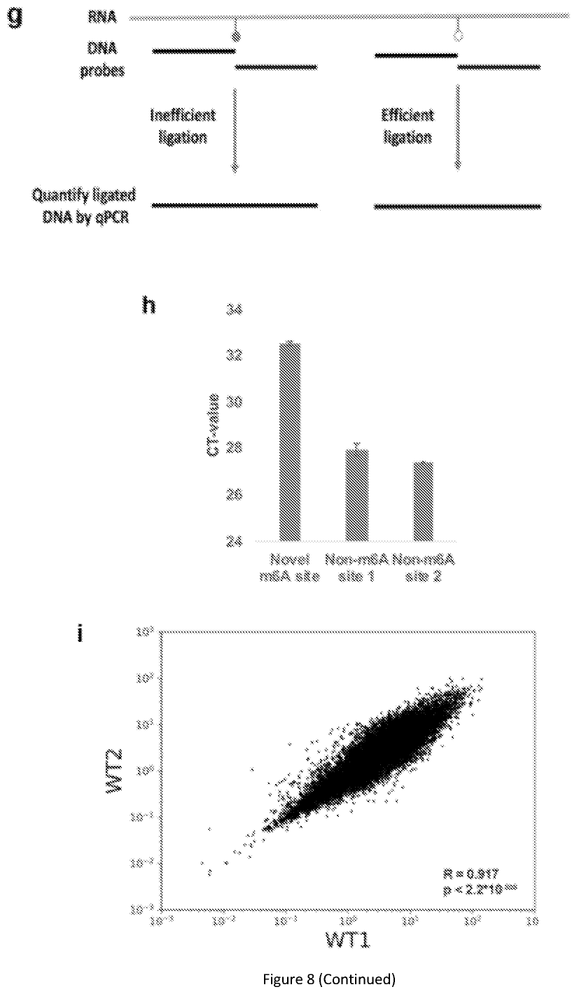

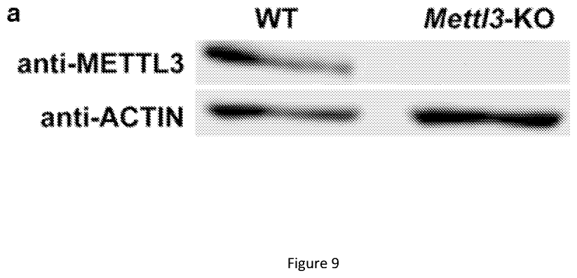

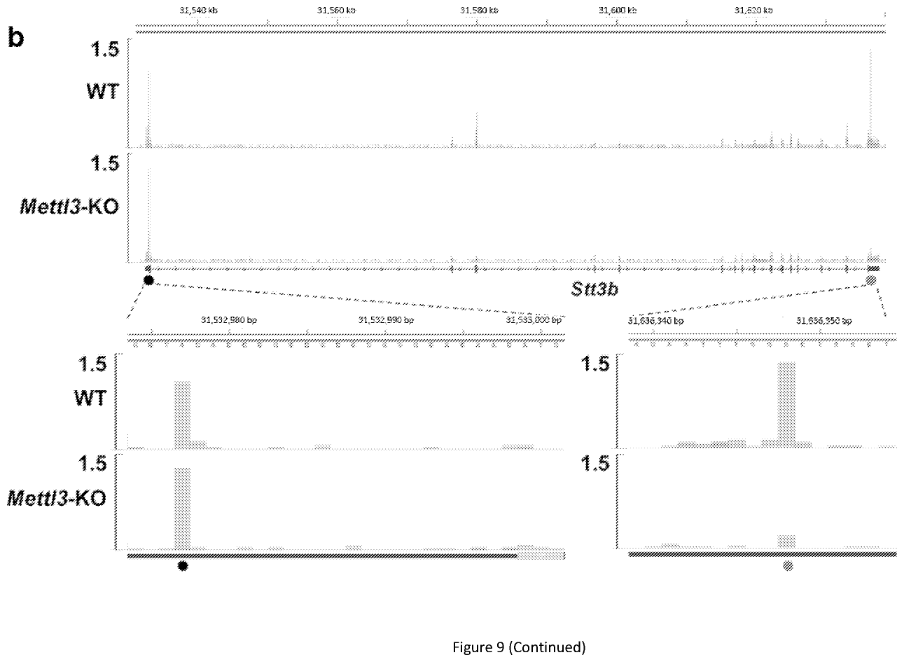

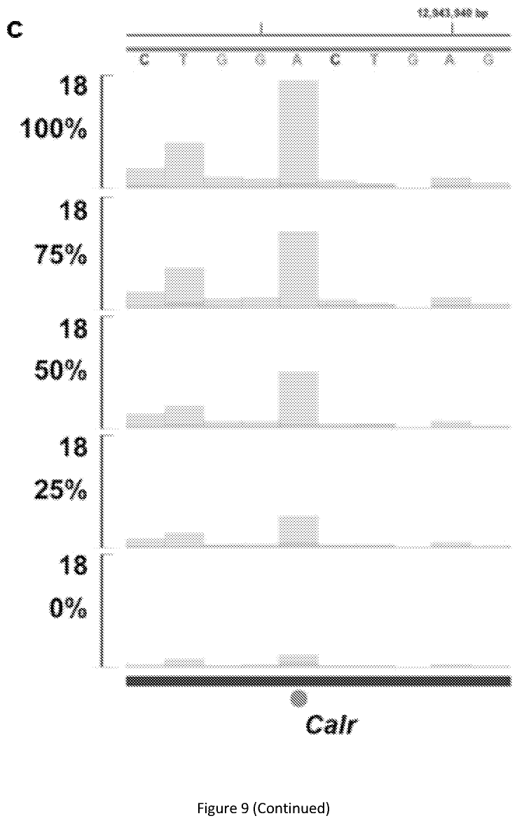

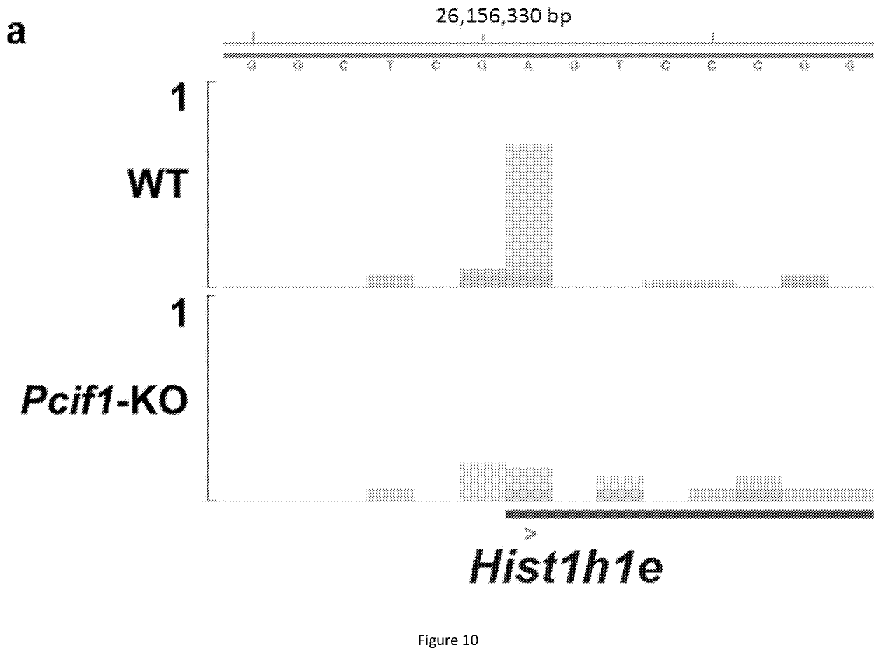

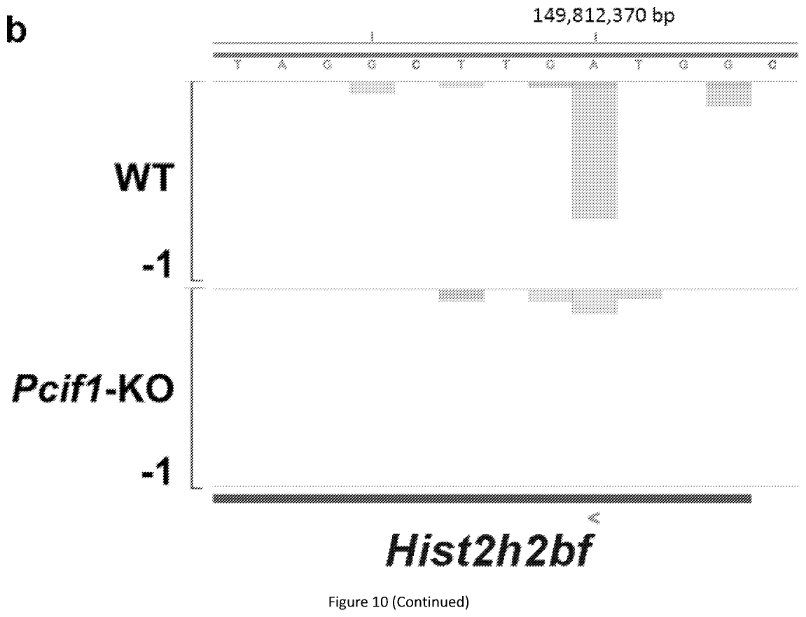

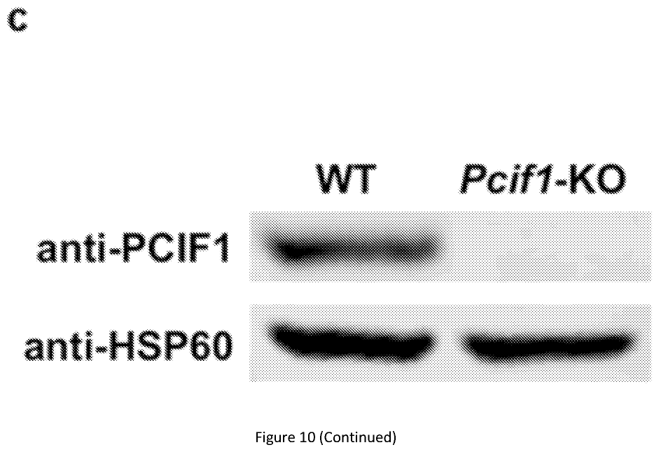

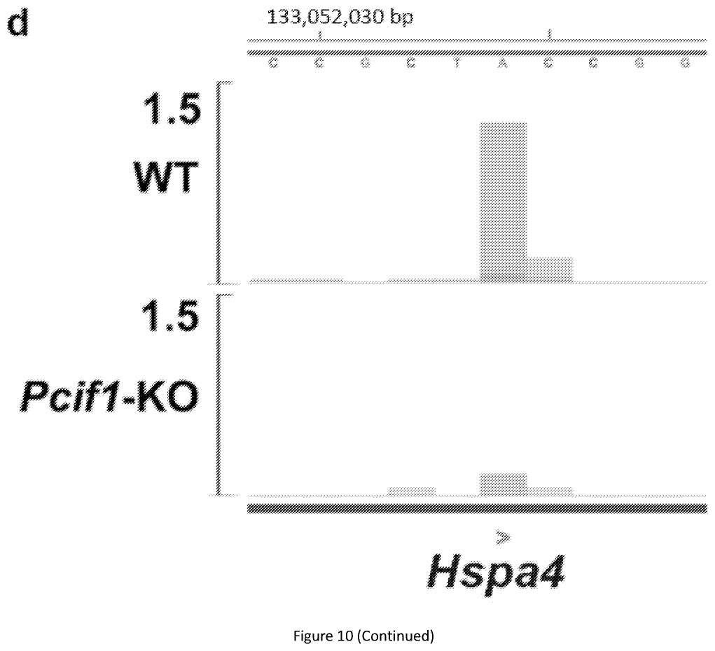

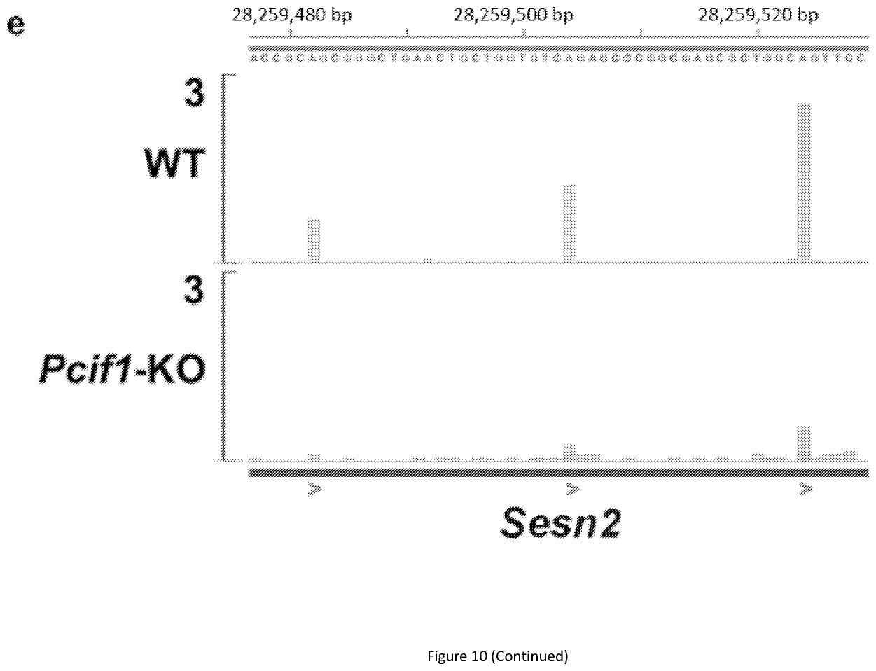

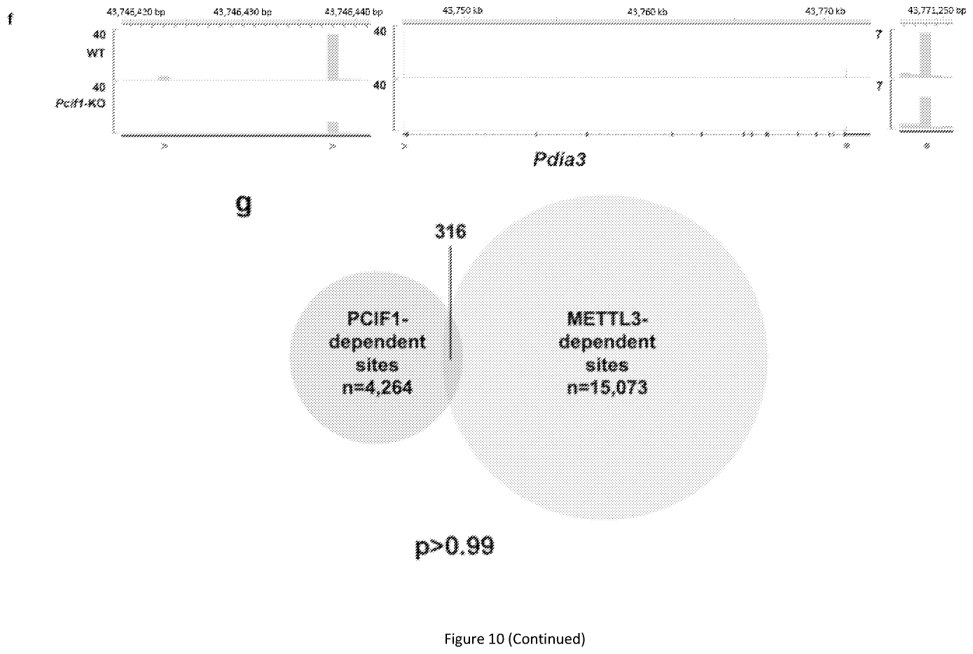

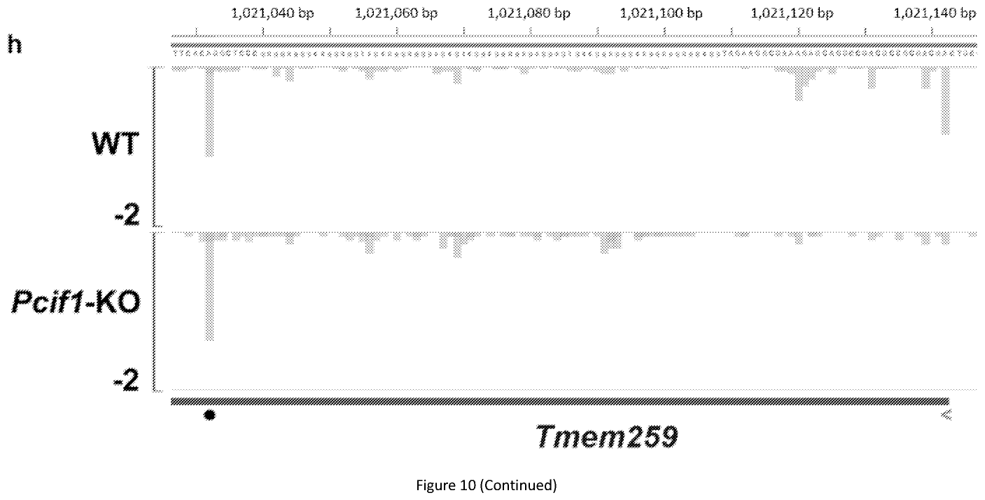

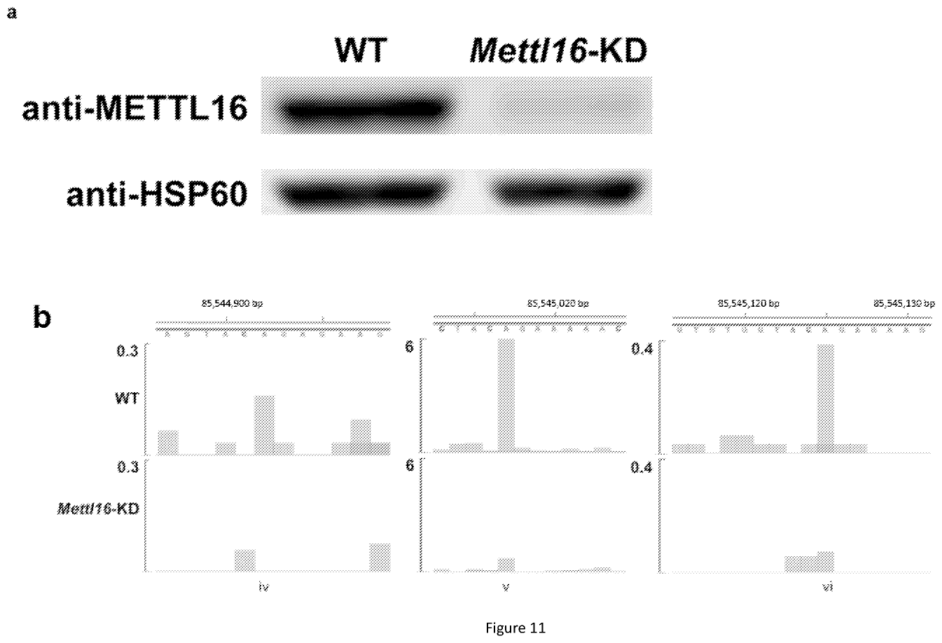

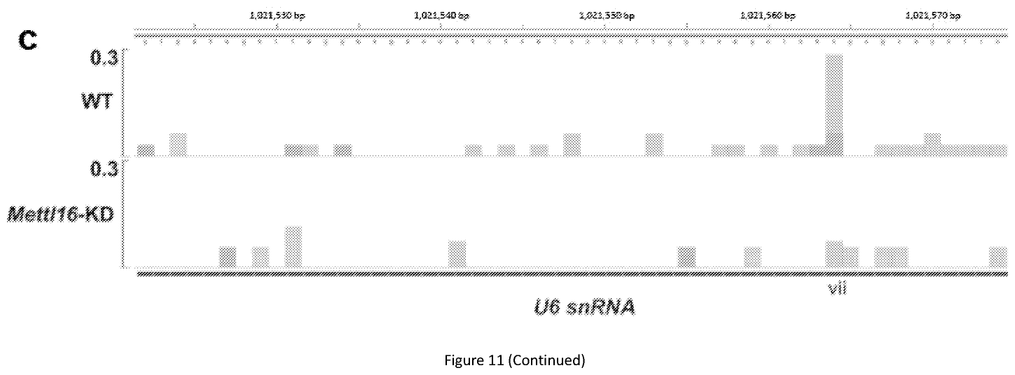

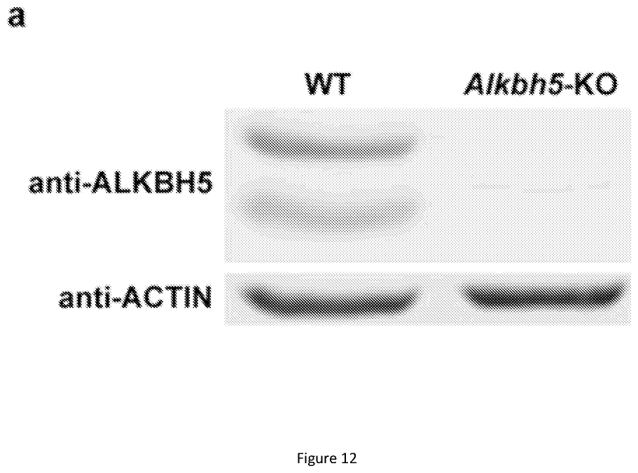

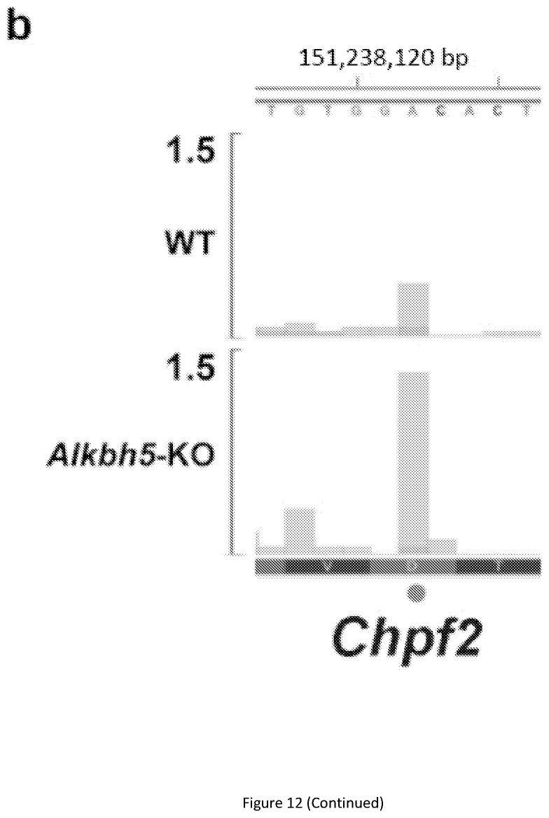

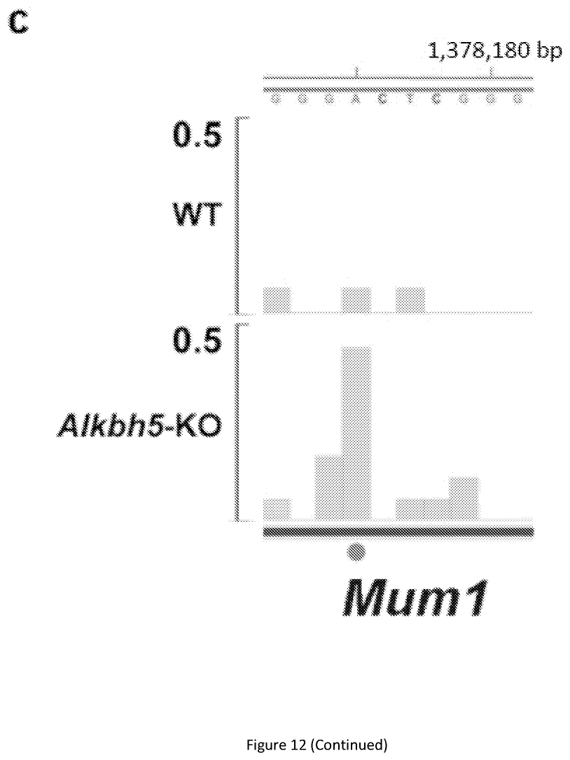

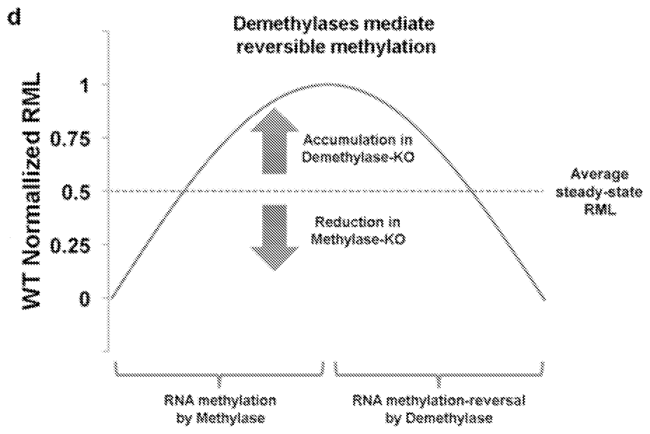

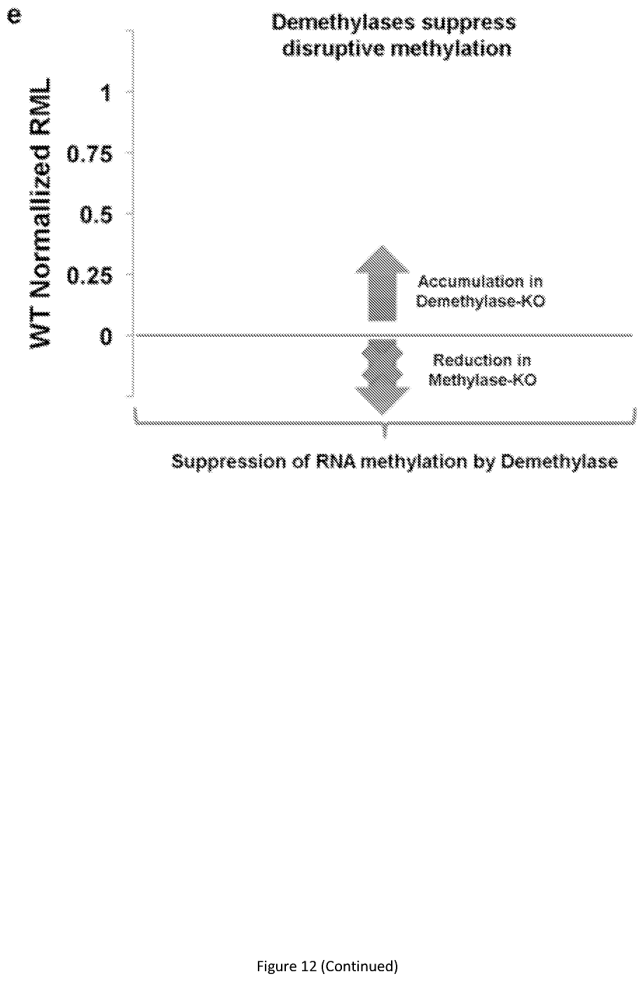

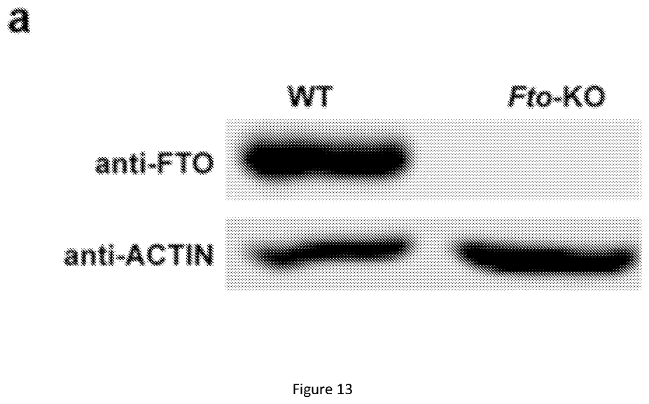

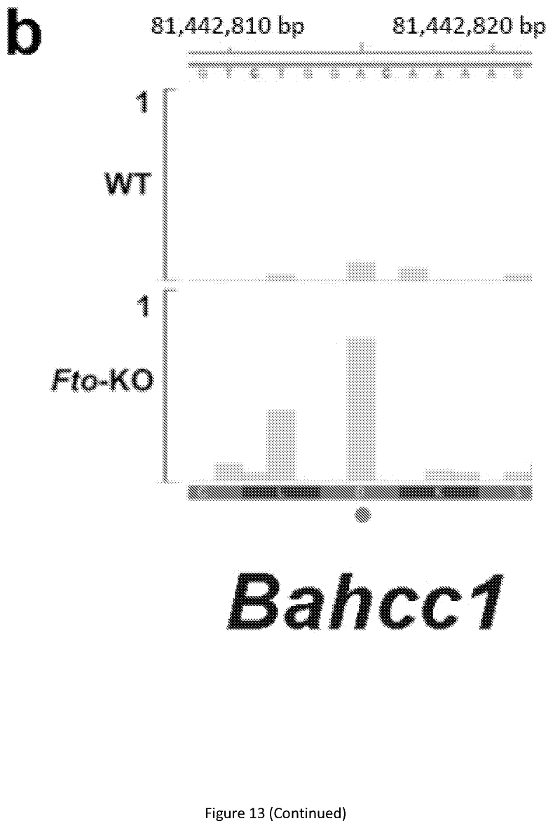

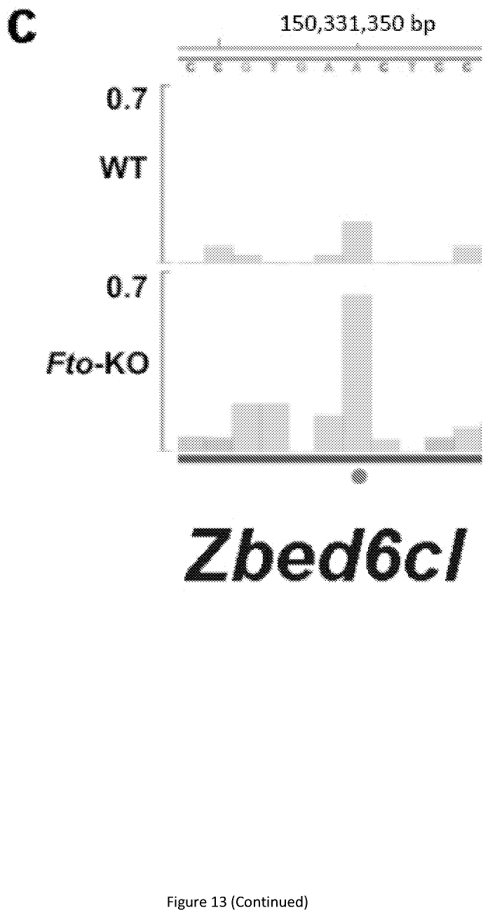

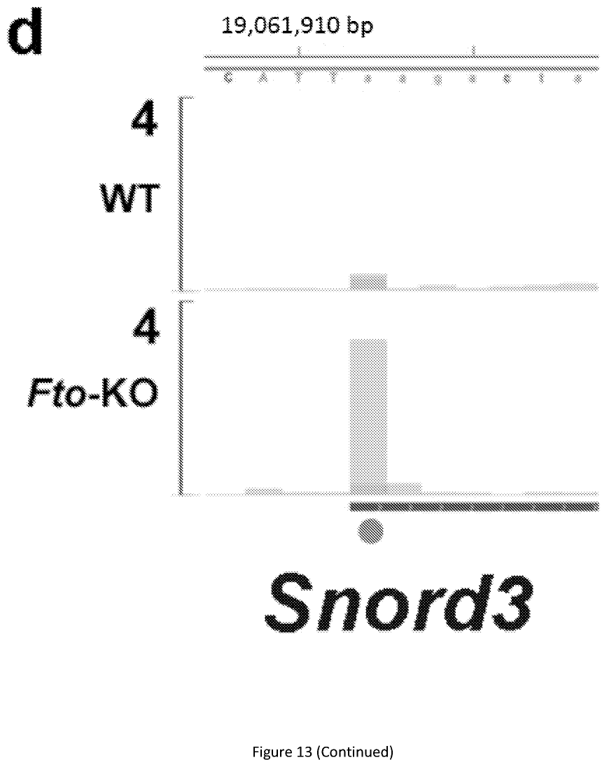

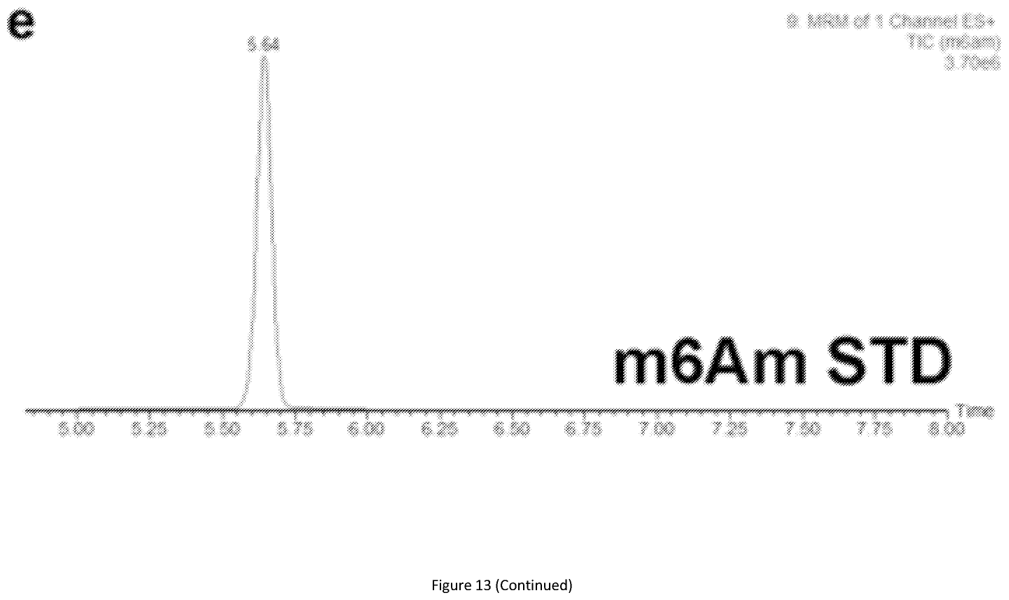

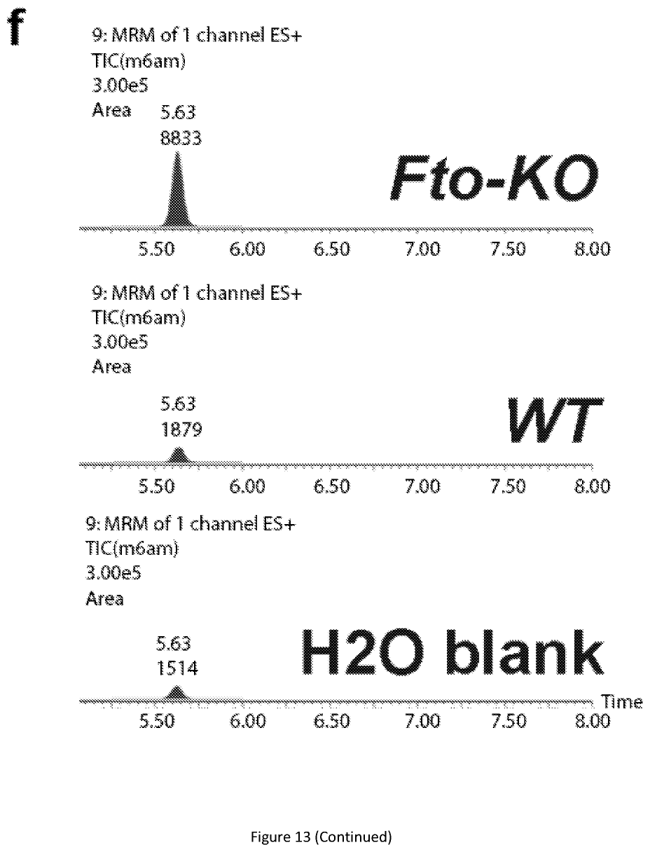

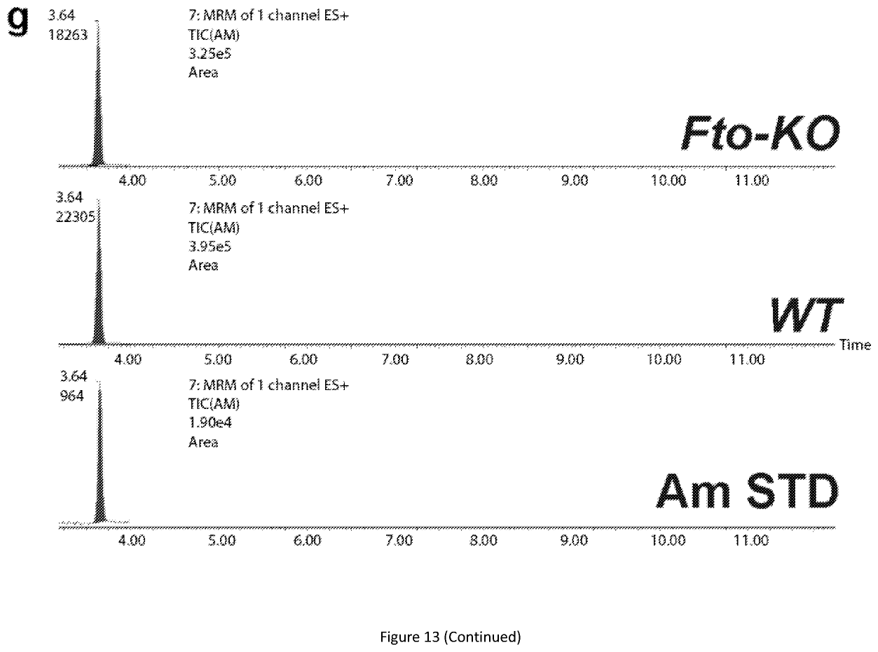

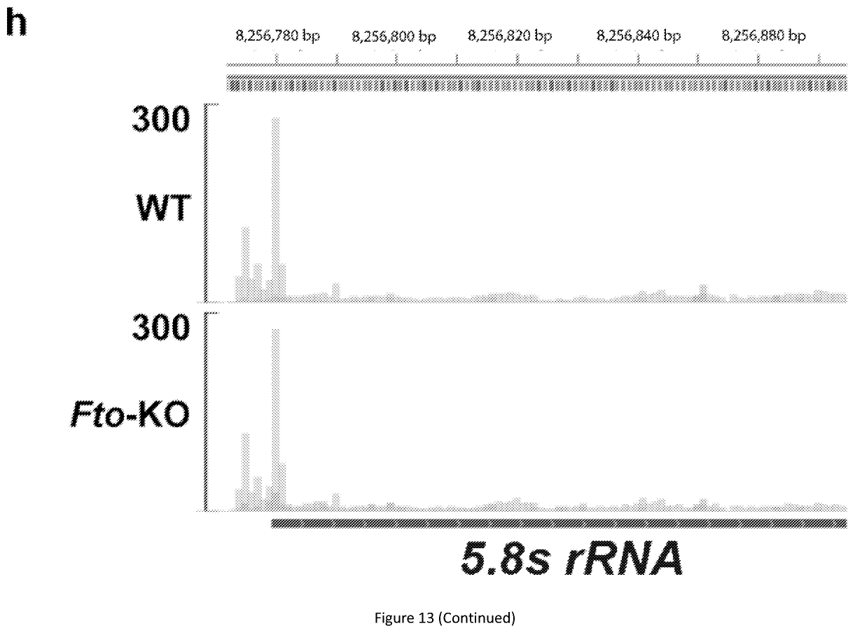

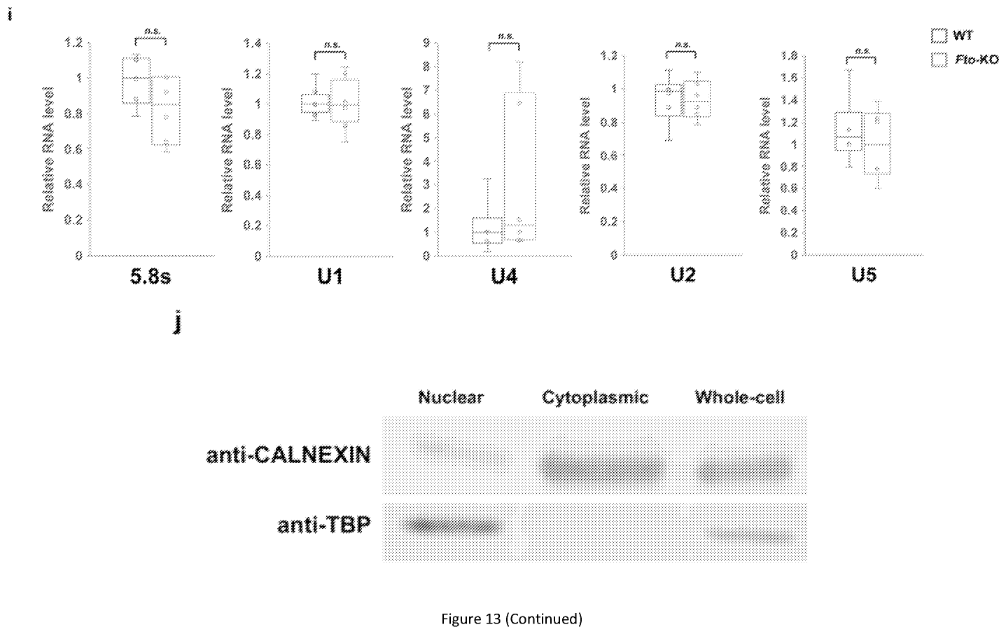

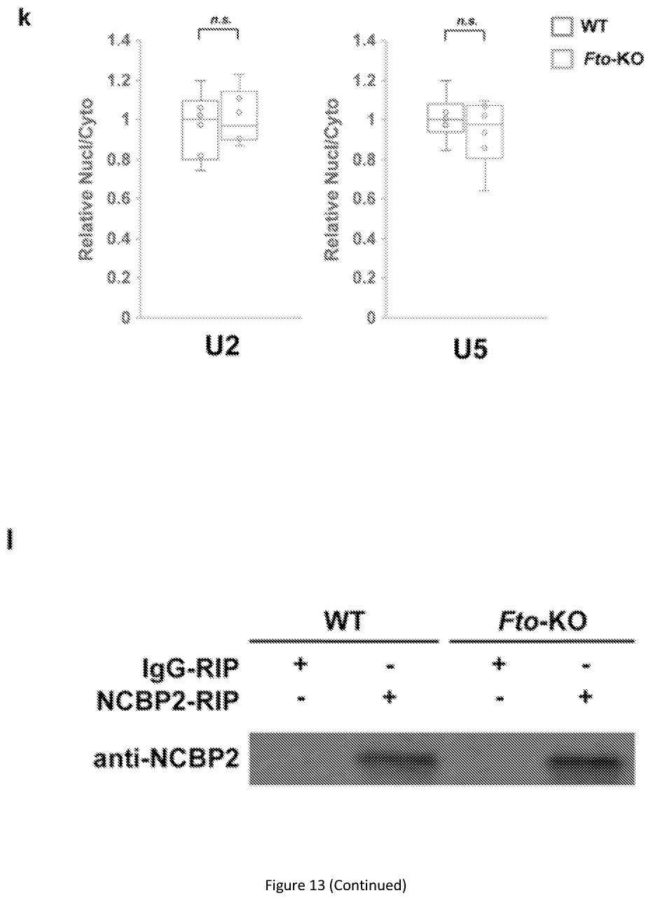

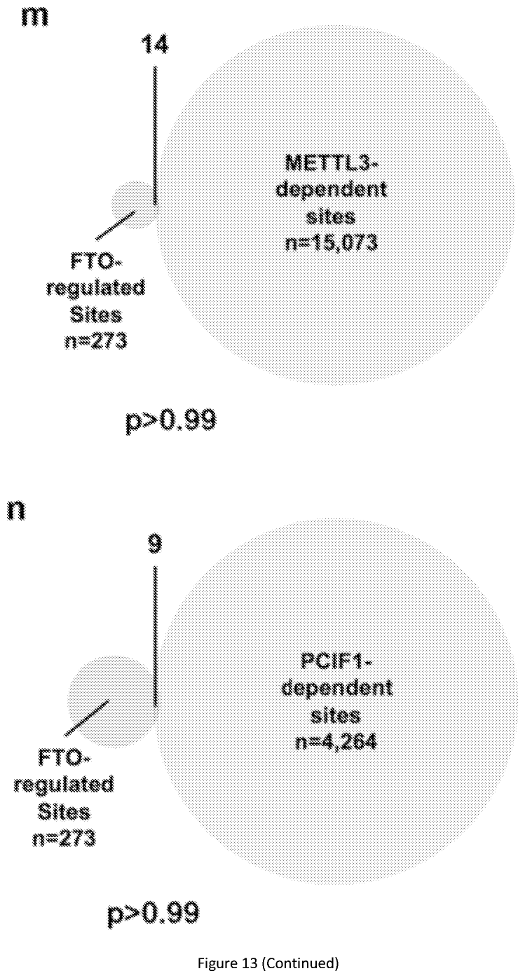

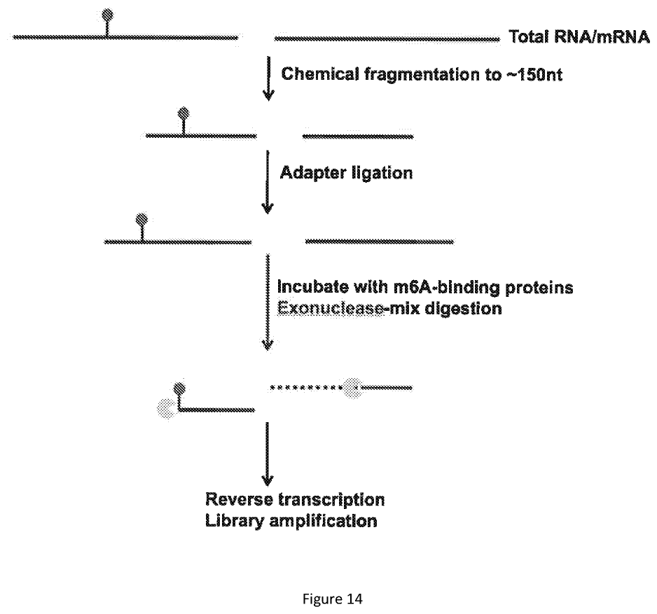

FIG. 9. m6ACE-seq maps RML reductions at METTL3-dependent m6A. (Related to FIG. 2).

(a) Western blotting demonstrating complete loss of METTL3 in Mettl3-KO cells.

(b,c) m6ACE (green) overlaid on Input (orange) read-start RPM counts mapped to a representative gene. METTL3-dependent and independent sites are respectively denoted by a red or black dot. Sequence corresponds to the same strand as the m6A site. Blue horizontal bars represent transcript models, with the green sections representing the start codon.

Magnified views of the 5′UTR and 3′UTR are also displayed for (B). For (C), percentages represent proportion of WT RNA in a mixture of WT and Mettl3-KO RNA.

FIG. 10. m6ACE-seq maps RML reductions at individual PCIF1-dependent m6Am sites. (Related to FIG. 3).

(a,b,d,e,h) m6ACE (green) overlaid on Input (orange) read-start RPM counts mapped to a representative 5′UTRs. PCIF1-dependent and PCIF1-independent sites are respectively denoted by red angles and black dots. Sequence corresponds to the same strand as the m6A/m6Am sites. Blue horizontal bars represent transcript models.

PCIF1-independent sites are sites where average RML reduction from WT to PCIF-KO is not significantly more than 1.5 fold (T-test p<0.05).

(c) Western blotting demonstrating loss of PCIF1 in Pcif1-KO cells.

(f) m6ACE (green) overlaid on Input (orange) read-start RPM counts mapped to a representative gene. PCIF1-dependent and METTL3-dependent sites are respectively denoted by red angles and red dots. Sequence corresponds to the same strand as the m6A/m6Am sites. Blue horizontal bars represent transcript models. Magnified views of the 5′UTR and 3′UTR are also displayed.

(g) Venn diagram representing overlap between PCIF1-dependent and METTL3-dependent sites. p-value of overlap by chance was calculated with a hypergeometric test.

FIG. 11. m6ACE-seq maps METTL16-dependent RML reductions at ‘UACAGAGAA’ sites within the Mat2a 3′UTR. Related to FIG. 4.

(a) Western blotting demonstrating loss of METTL16 in Mettl16-KD cells.

(b) Magnified representations of the Mat2a 3′UTR at positions iv, v and vi as denoted in (FIG. 3a).

(c) m6ACE (green) overlaid on Input (orange) read-start RPM counts mapped to U6 snRNA.

Sequence corresponds to the same strand as the m6A site, denoted by position vii. Blue horizontal bars represent U6 snRNA transcript.

FIG. 12. ALKBH5 demethylates both m6A and m6Am in vivo. (Related to FIG. 5).

(a) Western blotting demonstrating complete loss of ALKBH5 in Alkbh5-KO cells.

(b,c) m6ACE (green) overlaid on Input (orange) read-start RPM counts mapped to a representative CDS (b) or 3′UTR (c). ALKBH5-regulated sites are denoted by green dots.

Sequence corresponds to the same strand as the m6A sites. Blue horizontal bars represent transcript models.

(d,e) Simplified models for expected RML variations given a model where ALKBH5 mediates reversible RNA methylation in the cytoplasm (D) versus a model where ALKBH5 suppresses methylation from accumulating in the nucleus (e).

FIG. 13. FTO suppresses accumulation of disruptive methylation in specific RNAs. (Related to FIG. 6).

(a) Western blotting demonstrating complete loss of FTO in Fto-KO cells.

(b-d,h) m6ACE (green) overlaid on Input (orange) read-start RPM counts mapped to a representative CDS (b), 3′UTR (c) or sRNAs (d,h). FTO-regulated sites are denoted by green dots. Sequence corresponds to the same strand as the m6A/m6Am sites. Blue horizontal bars represent transcript models.

(e) Chromatogram of m6Am standard with retention time denoted.

(f,g) Chromatogram of m6Am (f) or Am (g) with peak areas and retention times denoted.

WT m6Am amounts are similar to that in H2O blanks and are thus in the non-quantifiable range (f).

(i) Box and whisker plots of various sRNA total levels relative to 7SL scRNA in WT (blue) and Fto-KO (orange) cells. Represented are median and interquartile ranges for 6 biological replicates, normalized to the corresponding median values in WT cells.

(j) Western blotting demonstrating enrichment of TBP (nuclear marker) and de-enrichment of CALNEXIN (ER/cytoplasmic marker) in nuclear lysate, as well as de-enrichment of TBP and enrichment of CALNEXIN in cytoplasmic lysate compared to whole-cell lysate.

(k) Box and whisker plots of ratio of sRNA levels (relative to 7SL scRNA) in nucleus over cytoplasm in WT (blue) and Fto-KO (orange) cells. Represented are median and interquartile ranges for 6 biological replicates, normalized to the corresponding median values in WT cells.

(l) Western blotting demonstrating specific pulldown of NCBP2 protein by anti-NCBP2 but not anti-IgG antibody in both WT and Fto-KO cells.

(m,n) Venn diagrams representing overlaps between FTO-regulated sites with METTL3-dependent m6A (m) or PCIF1-dependent m6Am (n). p-value of overlap by chance was calculated with a hypergeometric test.

n.s. denotes not significant.

FIG. 14. Schematic depicting workflow of m6ACE-seq.

### DEFINITIONS

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### DETAILED DESCRIPTION OF THE INVENTION

Having now generally described the invention, the same will be more readily understood through reference to the following examples which are provided by way of illustration, and are not intended to be limiting of the present invention.

The present invention relates to a method for analysis of methylation of ribonucleic acid (RNA) comprising the steps:

(i) contacting RNA with one or more antibodies which binds to methylated site(s) of RNA; wherein the methylated site(s) comprise at least one ribonucleotide base modified by one or more methyl groups;

(ii) photo-crosslinking the one or more antibodies to crosslink individual antibodies to the RNA molecule(s) to form RNA-antibody conjugates;

(iii) immunoprecipitating to separate the RNA-antibody conjugates;

(iv) treating the RNA-antibody conjugates with at least one exonuclease;

(v) removing the crosslinked antibodies from the RNA-antibody conjugates to release RNA; and

(vi) analysing the released RNA.

It will be appreciated that immunoprecipitating to separate the RNA-antibody conjugates may serve to enrich the RNA-antibody conjugates.

According to a further aspect, the method further comprises ligating first adapter nucleic acid molecules to the 3′ end of the RNA molecule(s). It will be appreciated that ligating first adapter nucleic acid molecules to the 3′ end of the RNA molecule(s) may be before or after treating with at least one exonuclease.

The first adapter nucleic acid molecules may be DNA adapters or RNA adapters. In particular, the sequence(s) of the first adapter nucleic acid molecules may be pre-determined. More in particular, the first adapter nucleic acid molecules may all have substantially the same sequence.

The method additionally comprises ligating second adapter nucleic acid molecules to the 5′ end of the RNA molecule(s) after treatment with exonuclease.

The second adapter nucleic acid molecules may be a RNA adapter. In particular, the sequence(s) of the second adapter nucleic acid molecules may be pre-determined. More in particular, the second adapter nucleic acid molecules may all have substantially the same sequence.

It will further be appreciated that any suitable ligase may be used to ligate the first and/or second adapter nucleic acid molecules. The ligases used for ligating the first and second adapter nucleic acid molecules may be the same ligase or different ligases. For example, a suitable RNA ligase may be used. Examples of a suitable RNA ligase include but are not limited to T4 RNA ligase or T4 RNA ligase 2.

It will further be appreciated that any suitable exonuclease may be used for the present invention. In particular, the exonuclease comprises a 5′ to 3′ exonuclease. For example, the exonuclease may be XRN1.

It will be appreciated that analysing the RNA includes identifying methylated sites.

For any aspect of the invention, analysing the RNA comprises reverse transcribing the released RNA to complementary deoxyribonucleic acid (cDNA) and analysing the cDNA.

The released RNA may be reverse transcribed using an oligonucleotide molecule complementary to the first adaptor molecule to form single stranded complementary deoxyribonucleic acid (cDNA). The single stranded cDNA may then be amplified by polymerase chain reaction (PCR) to form double stranded cDNA (ds cDNA). The PCR amplification may be with a first primer substantially complementary to the first adaptor molecule and a second primer substantially complementary to the second adapter molecule. The ds cDNA molecule may then be analysed.

Analysing the cDNA or ds cDNA comprises sequencing and/or mapping the cDNA or ds cDNA. It will be appreciated that any suitable sequencing and/or mapping method may be used.

### EXAMPLES

### Example 1: Materials and Methods

Tissue Culture

ATCC HEK293T CRL-3216 cells were cultivated in a sterile 5% CO2 incubator at 37° C., and in DMEM supplemented with 10% FBS and 1% penicillin/streptomycin. Cells within passage 3-20 were used for experiments. HEK293T cells were regularly subjected to MycoAlert Plus Mycoplasma kit (Lonza LT07) to verify that they were mycoplasma-free.

siRNA Knockdown

HEK293T cells were first seeded in a 6-well plate with a seeding density of 4.8*10⁵ cells per well. After 24 hrs, HEK293T cells were then transfected with 22 nM (50 pmoles/well) final concentration of respective siRNAs using RNAiMax transfection reagent (Invitrogen 13778) according to the manufacturer's instructions. siRNA s35507 (Thermo Scientific) was used to knockdown Mettl16. 24 hrs post-transfection, HEK293T cells were diluted to new plates at a seeding ratio of 1:9 to allow the cells to divide for an additional 48 hrs before being harvested for RNA or protein (total 72 hrs knockdown).

Generation of Knockout Cell Lines Using CRISPR-Cas9

HEK293T gene deletions were performed as previously described (Koh et al., 2018). Briefly, guide RNA sequences corresponding to a region around the start codon of the gene of interest were designed using CRISPOR, then cloned into pSpCas9 BB-2A-puro (Addgene 62988) plasmids. Pairs of guide RNAs (Table 1) were designed to induce either frameshift mutation close to the 5′end of the gene or to delete the start codon. HEK293T cells were plated in 12-well plates at 2*10⁵ cells/well in regular growth media but without antibiotics. 16-24 hr later, cells were transfected with 500 ng of each of the pair of guide RNA-expressing plasmid via Lipofectamine 2000 (Thermofisher 11668). 24 hr post-transfection, successfully transfected cells were selected via survival under a 72 hr treatment with 2 μg/ml puromycin. Puromycin-resistant cells were expanded for monoclonal dilution to select for monoclones that present desired gene deletions. The knockout mutations in these monoclones were further verified by loss of protein of interest via Western blotting.

Generation of Overexpression Cell Lines

Overexpression cell lines were generated by transfecting HEK293T cells with plasmids containing FL-FTO or ΔNLS-FTO (aa32-485) inserted upstream of a 3×Flag-tag. Cells were either harvested in 48 hrs for Flag-tagged protein extraction, or passaged after 24 hrs and expanded for an additional 48 hrs for RNA extraction or immunofluorescence. Fto-KO cells were transfected for Flag-tagged protein extraction while WT cells were transfected for RNA extraction and immunofluorescence.

TABLE 1
Sequences in this work

| Name | Sequence | Source |
|---|---|---|
| Methylated | UCUCCUUAGUAGCUCCUAAGm6AUCGUCGAGUUACACGACGACAUUGUUCCGACUGACG | Trilink |
| RNA spike-in | (SEQ ID NO: 1) | Biotechnologies |
| Mettl3 sgRNA | GAGAGTCCAGCTGCTTCTTG (SEQ ID NO: 2) | IDT |
| #1 | | |
| Mettl3 sgRNA | CTATATCCTGGAGCGAGTGC (SEQ ID NO: 3) | IDT |
| #2 | | |
| Pcif1 sgRNA | GCAGGGACGCTTCCTCCCGG (SEQ ID NO: 4) | IDT |
| #1 | | |
| Pcif1 sgRNA | AGGTCCTGAACCAGGCGGAT (SEQ ID NO: 5) | IDT |
| #2 | | |
| Alkbh5 sgRNA | CCTCATAGTCGCTGCGCTCG (SEQ ID NO: 6) | IDT |
| #1 | | |
| Alkbh5 sgRNA | ATAGTTGTCCCGGGACGTCA (SEQ ID NO: 7) | IDT |
| #2 | | |
| Fto sgRNA #1 | CTAAATCCCGTGGCGCTCGC (SEQ ID NO: 8) | IDT |
| Fto sgRNA #2 | AGCTTCGCGCTCTCGTTCCT (SEQ ID NO: 9) | IDT |
| 3′ Ligation | /5rApp/TGGAATTCTCGGGTGCCAAGG/3ddC/ (SEQ ID NO: 10) | IDT |
| DNA Adapter | | |
| 5′ Ligation | GUUCAGAGUUCUACAGUCCGACGAUCNNNNNNNB (SEQ ID NO: 11) | IDT |
| RNA Adapter | | |
| m6ACe | GCCTTGGCACCCGAGAATTCCA (SEQ ID NO: 12) | IDT |
| Reverse | | |
| Transcription | | |
| Primer | | |
| qPCR 7SL | ATCGGGTGTCCGCACTAAGTT (SEQ ID NO: 13) | IDT |
| scRNA for | | |
| qPCR 7SL | CAGCACGGGAGTTTTGACCT (SEQ ID NO: 14) | IDT |
| scRNA rev | | |
| qPCR 5.8S | GGTGGATCACTCGGCTCGT (SEQ ID NO: 15) | IDT |
| rRNA for | | |
| qPCR 5.8S | GCAAGTGCGTTCGAAGTGTC (SEQ ID NO: 16) | IDT |
| rRNA rev | | |
| qPCR U1 | CCATGATCACGAAGGTGGTTT (SEQ ID NO: 17) | IDT |
| snRNA for | | |
| qPCR U1 | ATGCAGTCGAGTTTCCCACAT (SEQ ID NO: 18) | IDT |
| snRNA rev | | |
| qPCR U2 | TTCTCGGCCTTTTGGCTAAG (SEQ ID NO: 19) | IDT |
| snRNA for | | |
| qPCR U2 | CTCCCTGCTCCAAAAATCCA (SEQ ID NO: 20) | IDT |
| snRNA rev | | |
| qPCR U4 | GCCAATGAGGTTTATCCGAGG (SEQ ID NO: 21) | IDT |
| snRNA for | | |
| qPCR U4 | CAAAAATTGCCAATGCCGACT (SEQ ID NO: 22) | IDT |
| snRNA rev | | |
| qPCR U5 | TGGTTTCTCTTCAGATCGCATAAA (SEQ ID NO: 23) | IDT |
| snRNA for | | |
| qPCR U5 | GCCAAAGCAAGGCCTCAAAA (SEQ ID NO: 24) | IDT |
| snRNA rev | | |
| qPCR Gapdh | GTCTCCTCTGACTTCAACAGCG (SEQ ID NO: 25) | IDT |
| for | | |
| qPCR Gapdh | ACCACCCTGTTGCTGTAGCCAA (SEQ ID NO: 26) | IDT |
| rev | | |
| Novel m6A | /5Phos/AACAGTTAAATTTACTCTATGGGCAGTCGGTGAT (SEQ ID NO: 27) | IDT |
| T3LIG probe L | | |
| Novel m6A | CCATCTCATCCCTGCGTGTCCCCTCTTTGGArCrU (SEQ ID NO: 28) | IDT |
| T3LIG probe R | | |
| Non-m6A 1 | /5Phos/AGTGTCCAAAGAGCTTCTATGGGCAGTCGGTGAT (SEQ ID NO: 29) | IDT |
| T3LIG probe L | | |
| Non-m6A 1 | CCATCTCATCCCTGCGTGTCCAGGTTTTTTCrCrU (SEQ ID NO: 30) | IDT |
| T3LIG probe R | | |
| Non-m6A 2 | /5Phos/ACTATGGGTGTTAAATCTATGGGCAGTCGGTGAT (SEQ ID NO: 31) | IDT |
| T3LIG probe L | | |
| Non-m6A 2 | CCATCTCATCCCTGCGTGTCCGCTTTTAGGCrCrU (SEQ ID NO: 32) | IDT |
| T3LIG probe R | | |
| Universal | ATCACCGACTGCCCATAGA (SEQ ID NO: 33) | IDT |
| qPCR primer | | |
| for T3 DNA | | |
| ligase assay | | |
| for | | |
| Universal | CCATCTCATCCCTGCGTGTC (SEQ ID NO: 34) | IDT |
| qPCR primer | | |
| for T3 DNA | | |
| ligase assay | | |
| rev | | |
| Methylated | CCGUCAAGGm6ACUGUCUCUGC (SEQ ID NO: 35) | Trilink |
| RNA for in | | Biotechnologies |
| vitro | | |
| demethylation | | |

m6ACE Library Preparation

Total RNA was first extracted using Trizol-LS (Ambion 10296) according to manufacturer's instructions, and further ethanol precipitated to remove residual salt. Poly(A) RNA was purified using Poly(A)Purist Mag kit (Thermofisher AM1922) according to manufacturer's instructions. After another round of ethanol-precipitation, poly(A) RNA was fragmented to 120-150 nt by incubating in RNA fragmentation buffer (Ambion AM8740). Fragmented RNA was ethanol-precipitated and dephosphorylated at its 3′ end with T4 PNK (M0201) in the absence of ATP, 5′ phosphorylation was then initiated by adding 1 mM ATP and incubating. 3′ adapters were ligated with truncated T4 RNA ligase 2 (NEB M0242). Ligated RNA was incubated with anti-m6A antibody. The antibody-RNA mixture was split into 50 μl aliquots and crosslinked with 254 nm UV radiation. The antibody-RNA mixture was recombined and 1% of it was set aside as “input” and the rest (designated as “m6ACE”) was immunoprecipitated on BSA-blocked Protein-A-dynabeads. m6ACE RNA was subjected to 5′ to 3′ exonuclease treatment with XRN-1 (NEB M0338). Both input and m6ACE RNA was reverse crosslinked in elution buffer (1% SDS, 200 mM NaCl, 25 mM Tris pH 8, 2 mM EDTA, 1 mg/ml Proteinase K (Thermo Scientific E00491). RNAs were ethanol-precipitated and ligated to 5′ adapters with T4 RNA ligase (Ambion AM2140). Reverse transcription was performed with SuperscriptIII (Invitrogen 18080). cDNA was used for PCR amplification with Phusion High-fidelity PCR mastermix (NEB M0530) ad Truseq PCR primers. Finally, primer-dimer and adapter-dimers were removed with AMpure XP beads before undergoing PE75 sequencing on the Illumina Miseq or Nextseq platforms (See FIG. 14).

Cellular Fractionation

Nuclear and cytoplasmic fractionation was performed using the Nuclei EZ prep nuclei isolation kit (Sigma NUC101) according to manufacturer's instructions. Purified intact nuclei and cytoplasmic lysates were subjected to either RNA or protein isolation.

RNA Isolation

Total RNA was isolated from adherent HEK293T using Trizol-LS (Ambion 10296) according to manufacturer's instructions and quantified using the Qubit RNA HS assay (ThermoFisher Q32855).

m6ACE Library Preparation

Poly(A) RNA was purified using Poly(A)Purist Mag kit (Thermofisher AM1922) according to manufacturer's instructions. Poly(A) RNA was fragmented using RNA fragmentation buffer (Ambion AM8740) and dephosphorylated at its 3′ end with T4 PNK (NEB M0201) for 30 min at 37° C. 5′ phosphorylation was then initiated by adding 1 mM ATP and incubating for 30 min at 37° C. 3′ ligation was then performed as previously described (Goh et al., 2015), where 5′adenylated,3-dideoxyC DNA adapters were ligated with truncated T4 RNA ligase 2 (NEB M0242) in 1×ATP-free T4 RNA ligase buffer [50 mM Tris pH 7.5, 60 μg/ml BSA, 10 mM MgCl2, 10 mM DTT, 12.5% PEG8000] for 2 hr at 25° C. 3′-ligated methylated RNA spike-in (Table 1) was added to ligated Poly(A) RNA and the mixture was denatured for 5 min at 65° C. before incubating for 2 min on ice. This denatured RNA mixture was incubated overnight at 4° C. with anti-m6A antibody (Synaptic Systems 202003) in 1×IP buffer [150 mM NaCl, 10 mM Tris pH 7.4, 0.1% IGEPAL CA-630 (Sigma 18896)] supplemented with RNasin Plus (Promega N2611). In parallel, Protein-A-dynabeads (Life Technologies 10002D) was blocked overnight at 4° C. in 1×IP buffer supplemented with 0.5 mg/ml BSA (Sigma A7906). The iced antibody RNA mixture was crosslinked with 0.15 J/cm² 254 nm UV radiation six times. Part of the antibody-RNA mixture was set aside as “input” and the rest (designated as “m6ACE”) was mixed with decanted BSA-blocked Protein-A-dynabeads for 1.5 hr at 4° C. Beads bound with crosslinked RNA were then washed with the following cold buffers in this order: Wash buffer 1 [1M NaCl, 50 mM HEPES-KOH pH 7.4, 1% Triton X-100, 0.1% Sodium Deoxycholate, 2 mM EDTA], Wash buffer 2 [0.5M NaCl, 50 mM HEPES-KOH pH 7.4, 1% IGEPAL, 0.1% Sodium Deoxycholate, 2 mM EDTA], Wash buffer 3 [1% Sodium Deoxycholate, 25 mM LiCl, 10 mM Tris pH 8, 1% Triton X-100, 2 mM EDTA], TE [10 mM Tris pH 8, 1 mM EDTA] and finally 10 mM Tris pH 8. m6ACE RNA was subjected to 5′ to 3′ exonuclease treatment with XRN-1 (NEB M0338) for 1 hr at 37° C. The m6ACE RNA-bead mixture was then washed with Wash buffer 1, Wash buffer 2, Wash buffer 3, TE and 10 mM Tris pH 8. Both input and m6ACE RNAs were reverse crosslinked in elution buffer [1% SDS, 200 mM NaCl, 25 mM Tris pH 8, 2 mM EDTA, 1 mg/ml Proteinase K (Thermo Scientific E00491)] for 1.5 hr at 50° C. RNAs were ethanol precipitated and ligated to 5′adapters (Table 1) with T4 RNA ligase (Ambion AM2140) for 16 hr at 16° C. Reverse transcription primer (Table 1) was annealed (72° C. 2 min, ice 2 min) and reverse transcription was performed with SuperscriptIII (Invitrogen 18080) for 1 hr at 50° C., with the reaction stopped by incubating for 15 min at 70° C. The cDNA was used for PCR amplification with Phusion High-fidelity PCR mastermix (NEB M0530) and Truseq PCR primers. Finally, primer-dimer and adapter-dimers were removed with AMpure XP beads before undergoing PE75 sequencing on the Illumina Nextseq platforms.

m6ACE Sequence Analysis

Fastq sequences were first trimmed of 5′ and 3′ adapter sequences and poly(A) tails using Cutadapt (Martin, 2011). The 8-mer ‘N7B’ (N=A/C/G/T, B=C/G/T) UMI located at the first 8 nucleotides of read 1 was registered and trimmed. Any complementary UMI sequence in read 2 was also trimmed. Reads were mapped to the methylated spike-in (Table 1) using Bowtie2, or to the hg38 assembly transcriptome (Gencode v28 comprehensive gene annotations) using STAR (Dobin et al., 2013; Langmead and Salzberg, 2012). Aligned pairs that had the same mapping coordinates and UMIs were filtered out as PCR duplicates. Read-start coordinates in hg38-mapped reads that began with an adenosine nucleotide, and had a minimum mean read count of 1 across the triplicate samples were collated. m6A or m6Am sites were identified as read starts that were at least 2-fold enriched in m6ACE libraries than in the corresponding input libraries. This enrichment was calculated using DESeq2 (Love et al., 2014) performed on A-only sites across triplicate pairs of m6ACE and corresponding input libraries (FDR<0.1, padj<0.05). Based on read-start patterns observed from m6ACE-seq of methylated spike-ins, we considered identified “sites” that were 1-4 nucleotides upstream of another identified significant Rm6AC site or “sites” found within clustered read-starts to be m6ACE-seq false-positives and filtered them out. To identify m6A or m6Am sites that were differentially methylated between sample conditions, we calculated the RML of each site in each sample: The read-start counts at positions −4 to 0 of each site in the m6ACE library were summed and divided by the read-start counts at positions −51 to 0 of the same site in the input library to give ‘X’. Similarly, the read-start counts at positions −4 to 0 of the spike-in m6A site in the m6ACE library were summed and divided by the read-start counts at positions −21 to 0 of the same spike-in m6A site in the input library to give ‘Y’. X was normalized to Y to give RML. RML values of each site was averaged across triplicates for each sample condition. A site was denoted as differentially methylated if the average RML differs between sample conditions with a log₂ fold-change (LFC) cutoff of 2.0 (for methylase-KO or demethylase-OE induced RML reduction) or 1.0 (for demethylase-KO induced RML accumulation), as well as a one-tailed T-test p-value cutoff of <0.05. Consensus motif analysis was performed using Meme-chip (Machanick and Bailey, 2011). Metagene analysis was performed using MetaPlotR (Olarerin-George and Jaffrey, 2017). Gene ontology analysis was performed using the PANTHER classification system (Mi et al., 2017). Probability of overlap of lists of m6A/m6Am sites were calculated using a hypergeometric distribution. ROCAUC analysis was performed as previously described with the following changes (Goh et al., 2010): the collection of all m6A and m6Am sites present in WT cells or exhibiting RML accumulation in demethylase-KO cells were ranked with the most insignificant site first, based on WT padj-value as calculated by DESeq2. An ROC curve was plotted based on the ability for a demethylase-regulated site (at LFC=0.0, 0.5, 1.0, 1.5; T-test p<0.05) to predict insignificant m6A/m6Am sites in WT cells, and the area under the curve was calculated.

T3 DNA Ligase Assay for m6A Detection

100 ng of DNAse-treated RNA was annealed with respective pairs of DNA probes (Table 1) in T3 DNA ligase buffer [66 mM Tris pH 7.6, 10 mM MgCl2, 1 mM ATP, 1 mM DTT, 7.5% PEG6000] by incubating for 3 min at 85° C. and 10 min at 35° C. 1 U of T3 DNA ligase was added and the reaction incubated for 15 min at 35° C. DNA probe ligation efficiency was quantified using quantitative PCR (qPCR) as described below.

RT-qPCR

Reverse transcription with real-time qPCR (RT-qPCR) was performed as previously described (Goh et al., 2014). Briefly, total RNA was treated with RQ1 DNAse (Promega M610A) according to manufacturer's instructions. RNA was then purified using Phenol chloroform-Isoamyalcohol (25:24:1) and precipitated with ethanol. Purified RNA was incubated with 125 ng of random hexamers and 0.5 ul of 10 mM dNTPs for 65° C. at 5 mins and placed on ice for at least 1 min. Reverse transcription was performed with 200 U of superscript III (Invitrogen 18080044) and incubated for 25° C. at 5 min, 50° C. at 1 hr and 70° C. at 15 min in a 20 ul reaction. 5 U of RNase H (Invitrogen 18020171) was added to each sample to digest remaining RNA for 20 mins at 37° C. cDNA was used for RT-qPCR with express SYBR greenER qPCR supermix (Invitrogen 11762100), according to manufacturer's instructions, and with respective forward and reverse primers as listed (Table 1).

Total Protein Isolation

Trypsinized HEK293T cells were washed twice with ice-cold PBS. Washed cells or intact nuclei were lysed in RIPA buffer [150 mM NaCl, 1% NP-40, 0.5% sodium deoxycholate 0.1% SDS, 50 mM Tris pH 8, 1× Complete Mini EDTA-free protease inhibitor] by tumbling for 30 mins at 4° C. Lysate was clarified by centrifuging at 16,000 g for 30 mins at 4° C. and protein concentration was quantified Pierce BCA protein assay kit (Thermo Scientific 23225).

Flag-Tagged Protein Purification

˜2*10⁷ transfected cells were trypsinized and washed twice with cold PBS. The cell pellet was then resuspended in 1 mL of freshly prepared lysis buffer [150 mM NaCl, 50 mM Tris pH 7.4, 1 mM EDTA, 0.1% IGEPAL, 1× Complete Mini EDTA-free protease inhibitor, 1× phosphatase inhibitor cocktail 2 (Sigma P5726)]. This mixture was pipette-mixed every 10 mins for a total of 30 mins before being clarified at 13,000 rpm for 30 mins at 4° C., and the supernatant was collected as the protein lysate, which was kept on ice until the purification step. 40 μl anti-FLAG M2 affinity gel (Sigma A2220) and 0.5 mL of equilibration buffer [150 mM NaCl, 50 mM Tris pH 7.4, 1 mM EDTA, 0.1% IGEPAL] were added to a SigmaPrep spin column (Sigma SC1000) and centrifuged at 1,000 g for 2 mins at 4° C. The flow-through was discarded and the column was washed by tumbling with 0.5 ml equilibration buffer for 10 min at 4° C. for a total of 3 washes. The protein lysate was added to the prepared column and tumbled for 4 hrs at 4° C. The unbound fraction was removed by centrifugation and the column was washed by adding 0.5 mL wash buffer [150 mM NaCl, 50 mM Tris pH 7.4, 1 mM EDTA, 0.1% IGEPAL, 1× Complete Mini EDTA-free protease inhibitor], and tumbled for 10 mins at 4° C., before centrifuging to remove the flow-through. This was repeated for a total of 3 washes. FLAG tagged proteins were then eluted by adding 100 μL of elution buffer [150 mM NaCl, 50 mM Tris pH 7.4, 1 mM EDTA, 1× Complete Mini EDTA-free protease inhibitor, and 0.15 μg/μL of 3× FLAG peptide (Sigma F4799)] to the column, tumbled for 30 mins before centrifugation into a new clean tube, for a total of 3 elutions. The final elution was performed at 13,000 g for 2 mins at 4° C. Glycerol was added to the purified proteins at a final concentration of 20% before snap-freezing and storing at −80° C.

In Vitro Demethylation Assay

Flag-tagged proteins were added to the assay buffer [50 mM KCl, 50 mM HEPES-KOH pH7.4, 100 μg/mL BSA, 2 μM MgCl2, 2 μM ascorbic acid, 0.2 μM (NH4)2Fe(SO4)2, 1 U/μL RNasin plus], with 0.4 μM final alpha-ketoglutarate and 0.4 μM methylated oligonucleotide (Table 1) in a total volume of 25 μL. The demethylation assay was performed at 25° C. for 2 hr, after which 1.25 μL of 1 mM EDTA was added to stop the reaction. 10 μL of 1% SDS and 1 μL of 200 μg/ml Proteinase K was added to the mixture and incubated for 1 hr at 56° C., shaking at 1,000 rpm. The RNA was then purified using Phenol-chloroform-Isoamyalcohol (25:24:1), precipitated with ethanol and eluted in H₂O.

Dot Blotting

Hybond+ membrane (GE RPN119B) was soaked in water for 10 mins before being secured into the manifold of the BIO-DOT apparatus (Biorad 1706545). Wells were washed with 400 μL H₂O. 5 pmol of methylated or unmethylated RNA standards (Table 1) were prepared and used per well. 5 μl 10× denaturing buffer [4M NaOH, 100 mM EDTA] was added to 45 μl samples or standards. 50 μL of denatured samples or standards were added to each well of the BIO-DOT apparatus and allowed to filter through. 400 μL of 1× denaturing buffer were added to all wells and filtered through. The membrane was taken out of the disassembled manifold, air-dried on filter paper for 5 mins at room temperature and subjected to UV to crosslink the RNA to the membrane in a Stratalinker (0.12 J), and allowed to rest for 30 secs before repeating for a total of 2 crosslinks. The membrane was blocked in Odyssey blocking buffer for 1 hr and stained overnight using anti-m6A antibody (Synaptic Systems 202003, 1:1000). The secondary staining protocol of dot blotting are the same as described for Western Blotting.

Western Blotting

Western blotting was performed as previously described (Koh et al., 2018) with the following antibodies and dilution scales used: 1 μg/ml Mouse anti-actin (Santa Cruz sc-8432); 200× diluted mouse anti-HSP60 (Abcam ab110312); 1,000× diluted rabbit anti-METTL3 (Bethyl Lab A301-567A-T), 1,000× diluted rabbit anti-METTL16 (Bethyl Lab A304-192A-T), 1,000× diluted rabbit anti-PCIF1 (Bethyl Lab A304-711A-T), 250× diluted rabbit anti-ALKBH5 (Sigma HPA007196), 1,000× diluted rabbit anti-FTO (Abcam ab126605), 1,000× diluted rabbit anti-CALNEXIN (Abcam ab22595), 2,000× diluted mouse anti-TBP (Abcam ab818), 2,000× diluted rabbit anti-NCBP2 (Abcam ab91560), 10,000× diluted IRDye 680RD goat anti-mouse IgG H+L (Licor 68070) and 10,000× diluted IRDye 800CW goat anti-rabbit IgG H+L (Licor 32211).

Immunofluorescence

Cells were seeded on poly-D-lysine coated coverslips (Neuvitro GG-14-PDL) in 24-well plates 24-48 hr before fixing. At ˜70% confluency, cells were washed in PBS once and fixed in 4% formaldehyde (diluted in PBS, Thermo 28906) for 10 mins. The cells were rinsed and washed thrice using cold PBS. All washes steps were done in 0.5 mL volume with shaking at 50 rpm for 5 mins at room temperature unless otherwise stated. Permeabilization of cell membranes was done using 0.1% Triton X-100 (diluted in PBS) for 10 mins. The cells were rinsed and washed thrice using PBS-T [PBS with 0.1% Tween 20]. Blocking was then done using PBS-T with 10% goat serum (Sigma G9023) for 1 hr at RTP. Primary antibodies used are rabbit anti-FTO (Abcam ab126605, 1:1,000) and mouse anti-FTO (Santa Cruz sc271713, 1:100), mouse anti-FLAG (Sigma F1804, 1:1,000), and were diluted in antibody dilution buffer [PBS-T with 1% goat serum]. The blocking solution was aspirated and the diluted primary antibody added directly to coverslip before incubating overnight in a 4° C. humid chamber. After which the cells were rinsed and washed thrice using PBS-T. Fluorescent secondary antibodies (Invitrogen A11019 & A11070) were also diluted in antibody dilution buffer and added directly to the coverslip before incubation in the dark for 1 hr at room temperature. The cells were rinsed and washed thrice using DPBS-T in the dark. Hoechst solution was prepared by diluting Hoechst 33342 (Invitrogen H3570) in DPBS. 10 mg/ml of Hoechst solution was used for nuclear staining in the dark for 5 mins. The cells were rinsed and washed thrice in DPBS in the dark for 10 mins each. The coverslips were then placed onto a drop of Prolong Diamond Antifade Mountant (Thermo P36970) on a glass slide, cured overnight and sealed with nail polish. Images were taken with a Leica DMi8 microscope.

snRNA Isolation and Nucleoside UHPLC-MS/MS

Nuclear-enriched RNA was resolved on a 6% TBE-urea gel then stained with SYBR-gold (Invitrogen S11494). U1/5.8s-rRNA and U4 snRNAs were purified by cutting out the 164 nt and 141 nt bands respectively, using the low-range ssRNA ladder as a size marker (NEB N0364). snRNAs were gel eluted in elution buffer [0.4M NaCl, 10 mM Tris pH 7.5, 1 mM EDTA pH 8] at 16° C. overnight with shaking at 2,000 rpm. RNA was then precipitated with equal volume isopropanol and washed with 70% ethanol before the pellet was dissolved in water. Up to 100 ng of each snRNA was decapped in a 10 μl reaction with 1× ThermoPol buffer (NEB B9004S) and 5 U RppH (NEB M0356) for 2 hr at 37° C. The reaction was supplemented with 2 μl 1 U/μl Nuclease P1 (Sigma N8630) in a 30 μl reaction with 0.2 mM ZnCl2 and 20 mM NH4OAc pH 5.3, incubated for 2 hr at 42° C. The reaction was finally supplemented with 2 μl 1 mU/μl phosphodiesterase (Sigma P3242) and 2 μl 1 U/μl alkaline phosphatase (Sigma P5931) in a 40 μl reaction with 100 mM NH4HCO3, incubated for 2 hr at 37° C., then heat inactivated for 5 min at 65° C. and subjected to UHPLC-MS/MS.

Nucleoside UHPLC-MS/MS was performed as previously described (Koh et al., 2018). Briefly, for reverse phase liquid chromatograph, a HSS T3 (1.8 μm; 2.1×100 mm) column was used with the following parameters. Mobile phase A: water+0.1% formic acid; mobile phase B: acetonitrile+0.1% formic acid; flow rate: 0.3 ml/min; column temperature: 40° C.; sample temperature: 4° C.; injection volume: 5 μl; sample loop: 5 μl. Elution gradient condition was set as 0 min 2% B, 0.5 min 2% B, 6 min 8% B, 6.5 min 8% B, 6.6 min 2% B, 8 min 2% B. Am and m6Am eluted with the retention times of 3.64 min and 5.64 min respectively. Tandem mass spectrometry was performed using a Xevo TQ-S, Waters machine with the following parameters. Ion mode: ESI positive; Acquisition model: MRM; capillary voltage: 3.2 kV; desolvation temperature: 400° C.; Con gas flow: 150 L/h; desolvation gas flow: 800 L/h; source temperature: 150° C.; collision energy: 16 eV. Am and m6Am were detected by monitoring mass transitions of m/z=282.27-136.0 and m/z=296.0-150.0 respectively. Sample Am or m6Am were quantified based on a linear calibration curve generated using Am (Berry & Associates PR3734) or m6Am (Carbosynth NM157470) nucleoside standards. All measurements were performed in technical triplicates.

Data Availability

Data were deposited in NCBI's Gene Expression Omnibus (GEO) under accession number GSE124509. The custom Python scripts used in this study are available on request. It will be appreciated that other applicable custom scripts may be developed accordingly.

### Example 2: Results

Briefly, anti-m6A antibodies were first photo-crosslinked onto m6A-containing RNA, which were thus protected from subsequent 5′ to 3′ exoribonuclease treatment. Sequencing of protected RNA fragments should theoretically reveal high-resolution detection of m6A locations (FIG. 1a). We first tested m6ACE-seq on a synthesized RNA oligonucleotide containing a single m6A nucleotide at position 21 (Table 1). Comparison of m6ACE reads to untreated input reads revealed a m6ACE-specific pileup of reads starting exactly at the m6A position (FIG. 1b). We also performed m6ACE-seq on RNA extracted from human HEK293T cells and focused on the 18S and 28S rRNA, which each have a single well-established m6A site (Piekna-Przybylska et al., 2008). Here, we observed the same m6ACE-specific pileup of read-starts at established 18S rRNA and 28S rRNA m6A positions (FIG. 1c-1d). This demonstrates that exonucleases are able to digest away RNA up to and including the nucleotide just 5′ of antibody-protected m6A nucleotide. Therefore, these m6ACE-seq profiles show that m6ACE treatment can be used to map exact locations of m6A within RNA.

m6ACE-Seq Accurately Maps m6A at Single-Base-Resolution Throughout the Human Transcriptome

We proceeded to explore the utility of m6ACE-seq to map m6A at a transcriptome-wide level by subjecting HEK293T polyA-selected RNA to m6ACE-seq. In order to assess the authenticity of m6ACE-seq identified m6A sites, we first focused on m6A sites that had previously been authenticated by an orthogonal sequencing-independent single-base-resolution m6A mapping technique known as SCARLET (N. Liu et al., 2013). Across the long non-coding RNA Malat1 and multiple mRNAs, m6ACE-seq read-starts exhibited sharp pileups at all 7 SCARLET-positive m6A sites and only 1 out of 6 SCARLET-negative sites (FIG. 8a-d). This further supports the sensitivity and specificity of m6ACE-seq. We noticed that read-starts sometimes form a clustered noise pattern at locations without established m6A sites (FIG. 8a). Close observation of SCARLET-positive sites also showed that besides the distinct pileup of read-starts at the exact site of m6A methylation, there is occasionally a mild pileup of read-starts at positions −4 to −3 of established m6A sites (FIG. 8a). Given that we had also noticed similar trends for m6ACE-seq reads within our synthesized m6A RNA oligonucleotide, 18S rRNA and 28S rRNA (FIG. 1b-1d), we factored these read patterns into our m6ACE-seq analysis for identifying m6A sites transcriptome-wide (see Methods). Consequently, m6ACE-seq identified 33,163 significant sites within the human transcriptome (false-discovery-rate, FDR<0.1, p<0.05). Within mRNA, m6A is known to localize preferentially within the CDS and 3′UTR proximal to the stop codon (Dominissini et al., 2012; Meyer et al., 2012). As expected, metagene analysis of m6ACE-seq-defined m6A sites recapitulated this localization (FIG. 1e). Consensus motif analysis of the sequences around all significant m6A sites also depicted a “DRm6ACH” motif, typical of human m6A sites (FIG. 1f; D=A/G/U) (Linder et al., 2015). m6A sites identified by m6ACE-seq also exhibited significant overlap with sites identified by previous single-base-resolution m6A-sequencing methods (FIG. 8e, hypergeometric test p<10-100) (Ke et al., 2017; Linder et al., 2015). We noticed a m6A candidate site in the mitochondrial 16S rRNA that was not identified in previous single-base-resolution methylomes (FIG. 8f). In order to validate this candidate site, we utilized a T3-DNA-ligase-based m6A detection assay, where DNA probes anneal to sequences flanking the queried site, and ligation efficiency of the probes is inversely correlated with methylation level at the site of query (FIG. 8g) (W. Liu et al., 2018). When compared to 2 separate 16S rRNA sites without strong m6ACE signals, ligation of probes flanking our candidate m6A site was ˜29-fold less efficient, thereby validating it as a novel m6A site (FIG. 8f,8h). Together, these findings show that m6ACE-seq is capable of single-base-resolution transcriptome-wide mapping of established and novel m6A sites.

m6ACE-Seq Quantitatively Maps RML Reductions at Individual PCIF1-Dependent m6Am Sites

We next sought to determine if m6ACE-seq can also map m6Am and focused on histone mRNAs that were previously shown to contain m6Am (Moss et al., 1977). We observed clear m6ACE-seq read-start pileups at or near the first nucleotide of histone mRNAs, indicative of m6Am (FIG. 10a,10b). We also observed strong m6ACE-seq read-start signals within mRNA 5′UTRs that remained undiminished in Mettl3-KO cells (FIG. 2b,9b). Given their localization near annotated transcription-start-sites (TSS) and their independence from Mettl3, these m6ACE-seq read-starts likely represent m6Am sites located in the first nucleotide right after the mRNA cap. Together, these highlight an additional utility of m6ACE-seq in that it can identify both m6A and m6Am.

PCIF1 has a predicted N6-methyladenine methylase domain and interacts with the phosphorylated C-terminal tail of RNA polymerase II during RNA transcription (Iyer et al., 2011). We subjected Pcif1-KO RNA to m6ACE-seq and found that PCIF1 depletion caused clear RML reductions in sites within 5′UTRs where m6Am resides (FIG. 3a, 3g,10a,10b,10d, 10f, 10g). If PCIF1 specifically catalyzes m6Am methylation adjacent to mRNA caps, we would also expect METTL3-dependent sites proximal to stop codons to be resistant to PCIF1 depletion. We tested this by focusing on mRNAs with both Pcif1-KO-induced RML reductions in 5′UTRs and METTL3-dependent m6As in the associated 3′UTRs. Indeed, the METTL3-dependent m6As in these mRNAs did not exhibit any RML reduction in PCIF1-KO cells (FIG. 3a,10f). We subsequently extended our analysis to identify all PCIF1-dependent sites, which amounted to 4,264 throughout the transcriptome (FIG. 3b). These sites were found in genes enriched for functional annotations that focused on RNA processing and mitochondrial translation processes (FIG. 3c). Unlike METTL3-dependent m6A, PCIF1-dependent sites recapitulated a mRNA localization shifted strongly towards the 5′UTR (FIG. 3d). PCIF1-dependent sites also exhibited a ‘CABU’ consensus motif that is a subset of the initiator consensus ‘BBCABW sequence found at TSSs, and is distinct from the DRm6ACH’ motif of METTL3-dependent m6A (FIG. 3e, CA′ denotes the TSS, B=C/G/U, W=A/U) (Vo ngoc et al., 2017). We also found no significant overlap in sites that are dependent on METTL3 versus PCIF1, further supporting PCIF1 as a methylase that mediates methylation of sites distinct from that of METTL3 (FIG. 10g). Together, these analyses validate the identity of these PCIF1-dependent sites as TSS-associated m6Am.

We next sought to determine what insights we can garner from utilizing single-base-resolution m6ACE-seq to map m6Am, which we would otherwise miss out if we instead used a low-resolution m6A-sequencing technique. For example, 5′UTRs can harbour both PCIF1-independent m6A and PCIF1-dependent m6Am. In fact, we found that more than a quarter of 5′UTRs that contain PCIF1-dependent m6Am also contain PCIF1-independent m6A (FIG. 3f). Closer observation of some of these 5′UTRs revealed that PCIF1-independent m6A can be quite proximal to PCIF1-dependent m6Am (FIG. 3g,10h). In such cases, a low-resolution m6A-sequencing method might not be able to detect m6Am methylation loss after PCIF1-depletion simply because of the proximal m6A, resulting in false-negatives.

An alternative way to map m6Am might be to simply identify annotated TSSs within broad methylated regions identified by low-resolution m6A-sequencing. To test the plausibility of such a strategy, we first compared how well our identified PCIF1-dependent m6Am aligned with TSSs identified previously via cap-analysis-gene-expression-sequencing (CAGE-seq) (Abugessaisa et al., 2017). Compared to all detected m6A/m6Am, PCIF1-dependent m6Am are indeed more enriched for TSS-alignment (FIG. 3h). However, there still exists a good proportion of m6Am sites that are slightly misaligned with respect to annotated TSSs, likely because of TSS heterogeneity (FIG. 3a,3g,10b,10d-10f) (Abugessaisa et al., 2017). This argues against the accuracy of using low-resolution m6A-sequencing to detect m6Am sites.

Furthermore, almost 700 5′UTRs contain multiple PCIF1-dependent m6Am sites, which is probably an underestimation as certain m6Am sites might be wrongly misannotated to be upstream of TSSs (FIG. 3h). Low-resolution m6A-sequencing will not be capable of detecting the individual m6Am sites in these 5′UTRs, leading to greater false-negative rates. Collectively, these findings demonstrate the importance of using single-base-resolution m6ACE-seq to accurately map m6Am.

METTL16 Depletion Reduces Methylation of a Plethora of m6A Sites Beyond its Direct ‘UACAGAGAA’ Targets

METTL16 is another m6A methylase that mediates m6A methylation in the ‘UACm6AGAGAA’ motif (Pendleton et al., 2017). Mat2a encodes a S-adenosyl-methionine (SAM) synthetase and its 3′UTR possesses 5 ‘UACAGAGAA’ sites. However, previous efforts to map m6A sites in this region with low-resolution m6A-RIP-seq yielded only 4 broad peaks dependent on METTL16 (Pendleton et al., 2017). To determine if m6ACE-seq could precisely map METTL16-dependent m6A sites, we knocked down (KD) Mettl16 expression and extracted cellular RNA for m6ACE-seq (FIG. 11a). Focusing first on the Mat2a transcript, we found clear Mettl16-KD-dependent reductions in read-start signals at all 5 ‘UACAGAGAA’ sites, and even at a similar ‘UACAGAAAA’ site within the Mat2a 3′UTR (FIG. 4a,4b,11b). We also observed Mettl16-KD-dependent read-start signals reductions located exactly at a previously established METTL16-dependent m6A site within U6 snRNA (FIG. 11c) (Pendleton et al., 2017). The ability of m6ACE-seq to map m6A in various sequence contexts also demonstrates that m6ACE-seq does not exhibit sequence bias restrictions in mapping m6A.

We extended our analysis to identify other METTL16-dependent sites and found 602 throughout the transcriptome (FIG. 4c). We note that METTL16 was previously shown to induce splicing of the Mat2a transcript and expression of MAT2A protein to synthesize SAM (Pendleton et al., 2017). As such, METTL16 depletion decreases MAT2A expression, resulting in reduced intracellular SAM, a key substrate required for RNA methylation. Therefore, loss of METTL16 can result in a transcriptome-wide loss of m6A methylation beyond sites directly methylated by METTL16. In support of this possibility, metagene analysis revealed that the identified METTL16-dependent m6A do not exhibit a localization pattern that is unique from that of the collection of all m6A sites (FIG. 1e,4d). Motif analysis also showed that sequences centred at METTL16-dependent m6A sites depicted the METTL3-dependent DRm6ACH′ motif rather than the METTL16-dependent ‘UACm6AGAGAA’ (FIG. 4e). In accordance with this, more than half of METTL16-dependent m6A were co-identified as METTL3-dependent m6A (FIG. 4f). A substantial portion of METTL16-dependent m6A were also co-identified as PCIF1-dependent m6Am (FIG. 4g). This suggests that METTL3 and PCIF1 are directly responsible for mediating methylation of identified METTL16-dependent sites. Together, these analyses argue that METTL16 mediates the methylation of the majority of METTL16-dependent m6A through an indirect mechanism, likely via controlling intracellular SAM levels.

ALKBH5 Suppresses Accumulation of m6A

While m6ACE-seq is able to quantitatively map methylase-dependent m6A/m6Am, we wanted to test if m6ACE-seq can also quantify loss of demethylation in demethylase-depleted cells. ALKBH5 is a Alkb family iron(II)/alpha-ketoglutarate-dependent dioxygenase that has a strong capacity to demethylate m6A (Zheng et al., 2013). Therefore, we depleted ALKBH5 and used m6ACE-seq to identify m6A with RML accumulation in Alkbh5-KO cells (FIG. 12a). As expected, we observed RML accumulation in individual DRm6ACH′ sites after ALKBH5-depletion (FIG. 5a,12b,12c). On a transcriptome-wide scale, we found 680 sites with RML accumulations after ALKBH5-depletion (FIG. 5b). ALKBH5-regulated sites were found to localize in a manner similar to stop-codon proximal METTL3-dependent m6A (FIG. 2c,5c). Motif analysis revealed that ALKBH5-regulated sites also exhibited a consensus ‘RRm6ACH’ motif similar to the METTL3-dependent DRm6ACH′ (FIG. 5d).

We subsequently enquired what the purpose of demethylating these ALKBH5-regulated sites is and envisioned two possibilities. First, these are dynamic sites that are initially methylated in the nucleus to allow for RNA-methylation-mediated regulation before being demethylated by ALKBH5 in the cytoplasm after said regulation has occurred (FIG. 12d). Second, methylation on these sites is to be avoided and thus, ALKBH5 acts to suppress these undesired modifications while the RNA is still in the nucleus so as to maintain them in a constantly unmethylated state (FIG. 12e). We reckoned that we could distinguish between the two possibilities by comparing the methylome dependent on METTL3 versus the methylome regulated by ALKBH5. Specifically, if ALKBH5-regulated sites are dynamic, they should exhibit a dynamic RML range that not only shows accumulation in ALKBH5-KO cells but also reduction in METTL3-depleted cells, allowing them to be co-identified as METTL3-dependent sites too (FIG. 12d). However, this is not the case as hardly any ALKBH5-regulated sites were co-identified as METTL3-dependent sites (FIG. 5e). We further reasoned that if ALKBH5-regulated sites are dynamic, they should on average exhibit a significant level of steady-state methylation in WT cells (FIG. 12d). To test this, we calculated the receiver-operator-characteristics-area-under-curve (ROCAUC) for how well ALKBH5-regulated sites were at predicting methylation absences at those very sites in WT cells. This revealed that the stronger a site is regulated by ALKBH5, the higher its likelihood of being unmethylated at steady-state in WT cells (FIG. 5f). As such, if not for their RML accumulations in Alkbh5-KO cells, the majority of ALKBH5-regulated sites would not even be identified to have the capacity to be methylated in WT cells. These findings argue against a model where ALKBH5-regulated sites are dynamic and instead suggest that ALKBH5 continuously acts on its regulated sites to keep them constantly and completely unmethylated in WT conditions (FIG. 12e).

FTO Loss Causes m6Am Accumulation that Disrupts Binding of Specific snRNA Precursors to Nuclear Export Machinery

FTO has also been shown to possess both m6A and m6Am demethylation activity in vitro but recent studies have reported conflicting results about its in vivo target (Darnell et al., 2018; Ke et al., 2017; Mauer et al., 2019; 2017; Rosa-Mercado et al., 2017; J. Wei et al., 2018; Zhao et al., 2018). To resolve this, we performed m6ACE-seq on Fto-KO RNA and identified 273 sites with RML accumulations as FTO-regulated sites (FIG. 6a,13a). Amongst these sites, we were able to find evidences of FTO-depletion induced RML accumulation at ‘DRACH’ sites within CDSs and 3′UTRs, typical of METTL3-dependent DRm6ACH′ (FIG. 13b,13c). This supports FTO to be capable of demethylating internal m6A sites within mRNAs throughout the transcriptome.

Compared to mRNAs, transcripts that exhibited the greatest RML accumulations in FTO-KO cells were the small RNAs (sRNAs), specifically small nucleolar RNAs (snoRNA) and small nuclear RNA (snRNA) (FIG. 6a). Upon close inspection of these sRNAs, we observed that the exact site of significant RML accumulation generally matched the first nucleotide of each sRNA (FIG. 6b,6c,13d). The first nucleotide of Sm-class snRNAs are also known to be methylated at the 2′-O-ribose (Karijolich and Yu, 2010). Therefore, the RML accumulation at the first nucleotide of snRNAs ought to represent reduced N6-demethylation of m6Am. To test this, we used ultra-high-performance-liquid-chromatography coupled with tandem mass spectrometry (UHPLC-MS/MS) to quantify Am and m6Am levels, focusing on U1 and U4 nRNAs as these snRNAs exhibited robust RML accumulations upon Fto-KO (FIG. 6a-6c). While there was no quantifiable snRNA m6Am in WT cells, there were indeed significant m6Am levels for both snRNAs in Fto-KO cells (FIG. 6d,13e-13g).

Previous studies reported that m6Am accumulation in the absence of FTO leads to increased levels of the m6Am-containing RNAs (Mauer et al., 2019; 2017). Therefore, we would expect RNA levels for U1 and U4 snRNA to be higher in Fto-KO cells, especially in comparison to a sRNA like 5.8s rRNA, which has no m6Am (FIG. 13h). However, Fto-KO did not result in any significant changes in the relative RNA levels of any snRNA (FIG. 13i). Since the maturation of Sm-class snRNAs involves translocation from the nucleus to the cytoplasm and back to the nucleus again (Kiss, 2004), we performed cell fractionation to isolate nuclear and cytoplasmic fractions and quantified if any of the Sm-class snRNAs displayed a shift in their cellular localizations (FIG. 13j). We found that U1 and to a greater degree, U4 snRNAs were more confined to the nucleus in Fto-KO cells (FIG. 6e). A previous study reported U2 and U5 snRNAs to be targeted by FTO (Mauer et al., 2019). While we also found both snRNAs to exhibit RML accumulations in the first nucleotide, the accumulations were neither as significant nor as robust as those of U1 and U4 snRNAs (FIG. 6a). Likewise, U2 and U5 snRNAs both did not exhibit any shifts in cellular localization similar to that of U1 and U4 snRNAs (FIG. 13k).

The increased nuclear localization of specific snRNAs could be caused either by inhibition of nuclear export of the initial precursor snRNA or improved nuclear import of the mature snRNA (Kiss, 2004). Sm-class snRNA precursors possess 5′ caps that bind to NCBP2 within the cap-binding complex, which mediates export of bound capped transcripts out of the nucleus, so we tested if m6Am accumulation affects snRNA binding to NCBP2. Using NCBP2 RIP (FIG. 13l), we found that NCBP2 binding to Fto-KO U4 snRNA was ˜5-fold weaker than to WT U4 snRNA (FIG. 6f). Therefore, in the absence of FTO, m6Am methylation accumulation in the first nucleotide of U4 snRNA potentially disrupts binding of the adjacent snRNA cap to NCBP2. This results in decreased nuclear export of U4 snRNA precursor and increased nuclear retention.

We next enquired if FTO suppresses methylation of its regulated sites (FIG. 12e) or if it mediates methylation-reversal as previously reported (FIG. 12d) (Mauer et al., 2019; 2017; J. Wei et al., 2018). Similar to ALKBH5-regulated sites, hardly any FTO-regulated sites were co-identified as methylase-dependent sites (FIG. 13m,13n). Furthermore, ROCAUC analysis revealed that the degree of FTO-mediated demethylation is strongly predictive of a FTO-regulated site being unmethylated in WT cells at steady state (FIG. 6g). Therefore, FTO acts to keep its regulated sites constantly and completely unmethylated.

FTO Overexpression Causes Aberrant mRNA Methylation-Suppression in the Nucleus

FTO is mainly localized to the nucleus (Jia et al., 2011). However, recent work reported the detection of cytoplasmic FTO, arguing that FTO can thus mediate RNA methylation-reversal in the cytoplasm (Aas et al., 2017; J. Wei et al., 2018). We sought to validate FTO's cellular localization using a specific antibody and found FTO to be strictly nuclear-localized (FIG. 7a; anti-FTO i). Furthermore, we found that another antibody previously used to demonstrate FTO's cytoplasmic localization by immunofluorescence was actually non-specific (FIG. 7a; anti-FTO ii) (Aas et al., 2017). This highlights the absolute necessity of using Fto-KO cells to validate FTO's cellular localization.

We next tested if WT-FTO overexpression (OE) caused any aberrant RNA demethylation. Through m6ACE-seq of FTO-OE RNA, we found that FTO overexpression indeed caused m6A reductions at 373 sites transcriptome-wide (FIG. 7b-e). These sites tend to exhibit a ‘DRm6ACW’ motif (FIG. 7f). Notably, none of the sites that exhibit RML accumulation in Fto-KO cells were affected by FTO overexpression. This was expected given that endogenous-FTO-regulated sites generally have no steady-state methylation in WT cells and thus cannot be aberrantly demethylated by exogenous FTO (FIG. 6g). Since the overexpressed FTO is strictly nuclear, the demethylation observed is likely overexpressed FTO aberrantly suppressing m6A methylation of mRNA before it shuttles out of the nucleus (FIG. 7b).

### Example 3: Discussion

Various methylases and demethylases catalyze methylation and demethylation of N6methyladenosine (m6A) and N6,2′-O-dimethyladenosine (m6Am) but precise methylomes uniquely mediated by each methylase/demethylase are still lacking.

We describe here a novel technology for transcriptome-wide sequencing of the epitranscriptomic RNA modifications: m6A and m6Am at single nucleotide resolution. We term our technology m6A-Crosslinking-Exnuclease-sequencing (m6ACE-seq).

Anti-m6A polyclonal antibody is first crosslinked onto m6A/m6Am RNA using 254 nm ultraviolet radiation. Input RNA is then set aside at this point. Subsequently, the remainder RNA-antibody complexes are immunoprecipitated and subjected to 5′-to 3′ exonuclease treatment. The crosslinked antibody protects RNA downstream of any m6A or m6Am from being digested by the exonuclease, resulting in RNA fragments containing m6m or m6Am in the first nucleotide. These fragments are then sequenced on IIlumina platforms. It will be appreciated that other sequencing platforms may be used.

Normalization of immunoprecipitated+digested RNA fragment levels against the respective input RNA fragment levels also provides a relative quantification of the RNA methylation level.

We developed a novel technique for quantitative single-base-resolution sequencing of m6A and m6Am that overcomes the technical limitations of past methods. We used m6ACE-seq to precisely map transcriptome-wide locations of m6A/m6Am in cells individually depleted of each and every known catalytic methylase or demethylase, generating a comprehensive atlas of m6A/m6Am methylome maps that are regulated by each specific methylase or demethylase. Comparisons of distinct methylomes allowed us to redefine the purpose of demethylase activity, thereby highlighting the utility of our technique in investigating the regulation and function of m6A and m6Am.

Here, we developed m6A-Crosslinking-Exonuclease-sequencing (m6ACE-seq) to map m6A and m6Am at transcriptome-wide single-base-resolution. m6ACE-seq's ability to quantify relative differences in methylation levels across samples enabled the generation of a comprehensive atlas of distinct methylomes uniquely mediated by every individual known methylase/demethylase. Our atlas revealed METTL16 to indirectly impact manifold methylation targets beyond its consensus target motif and highlighted the importance of precision in mapping PCIF1-dependent m6Am. Rather than reverse RNA methylation, we found that both ALKBH5 and FTO demethylases instead maintain their regulated sites in an unmethylated steady-state. In FTO's absence, anomalous m6Am disrupts snRNA interaction with nuclear export machinery, potentially causing aberrant pre-mRNA splicing events. We propose a model whereby RNA demethylases ensure normal RNA metabolism by suppressing disruptive RNA methylation in the nucleus.

By photo-crosslinking specific antibodies, we were able to induce a tight protection of m6A/m6Am and its downstream RNA from 5′ to 3′ exoribonucleases. Sequencing of this protected RNA fragment as read-start pileups allowed us to map RNA methylation at single-base-resolution. We confirmed this property by precisely mapping known m6A locations in synthesized RNA oligonucleotides and m6A sites in multiple human RNAs that were previously established using an orthogonal approach (N. Liu et al., 2013). We also demonstrated that m6ACE-seq is capable of mapping m6Am found adjacent to the mRNA cap. Compared to CLIP-based m6A-sequencing methods (see Table 2), m6ACE-seq does away with inconvenient radioactive gel electrophoresis steps and effectively halves the time needed for library preparation (Ke et al., 2017; 2015; Linder et al., 2015). Since m6ACE-seq maps methylation based on the pileup of read-starts as opposed to mutational signatures, m6A mapping is also less sensitive to small nucleotide polymorphisms or any random sequencing errors. The 5′ adapter that we used to directly ligate to the methylated RNA terminates with a 3′ 8-mer UMI. Besides allowing us to eventually correct for amplification bias, this randomized UMI also avoids any ligation bias or mispriming artifact that might wrongly bias certain RNA sequences to be mapped as methylated. For identifying m6A or m6Am, we normalized m6ACE-induced read-start pileups against random-fragmentation-induced read-start pileups in a RNA input library constructed in parallel using the same RNA with the exact same library construction steps except without immunoprecipitation and exoribonuclease treatment (FIG. 1a). This normalization strategy is also used for m6A-RIP-seq but is generally absent from CLIP-based m6A-sequencing methods and analyses. Finally, we integrated methylated RNA spike-ins to correct for differences in crosslinking/immunoprecipitation/exonuclease efficiencies between samples. Overall, these steps allow m6ACE-seq to quantify relative differential methylation levels across sample types.

TABLE 2
Comparison of various m6A sequencing technologies

| | Resolution | Prep-time | |
|---|---|---|---|
| Method | (nt) | (days) | Complications |
| m6A-RIP-seq | 100-200 | 3 | Poor resolution |
| PA-m6A-seq | ~23 | 5-6 | Requires 4-ThioU |
| miCLIP | 1 | 10 | Requires |
| or | | | radioactivity and |
| m6A-CLIP | | | multiple PAGE |
| | | | purifications |
| M6ACE | 1 | 4 | |

Using m6ACE-seq on cells individually depleted of every known methylase or demethylase, we generated the first comprehensive atlas of single-base-resolution methylomes unique to each individual m6A/m6Am methylase or demethylase. This atlas afforded us the ability to compare methylomes specific to distinct methylases, and has helped to tackle previously unanswered questions. For example, despite exhibiting a strong specificity to methylate only ‘UACAGAGAA’ motifs, a previous report had used low-resolution m6A-RIP-seq and found that a majority of METTL16-dependent methylated regions lack any ‘UACAGAGAA’ (Pendleton et al., 2017). METTL16 was speculated to either directly methylate these regions through an unknown co-factor that directs METTL16 to non-‘UACAGAGAA’ sequences, or indirectly through modulating intracellular SAM levels, which is a substrate required for RNA methylation. Comparison of METTL3- and PCIF1-dependent methylomes with METTL16-dependent methylomes at single-base-resolution allowed us to conclude in support of the latter indirect mechanism. METTL16's ability to modulate the methylation and consequently, the processing of a plethora of m6A sites beyond its direct targets is perhaps a contributing reason to why it is an essential gene in mammalian cells (Pendleton et al., 2017). We also focused on PCIF1, which other groups validated as a m6Am methylase during the preparation of our manuscript (Akichika et al., 2018; Sun et al., 2018). However, our study is unique for our single-base-resolution methylome of PCIF1-dependent m6Am sites, allowing us to find that TSS heterogeneity, m6Am clustering or proximal PCIF1-independent sites can easily result in false-negatives. This emphasizes the need for pinpoint precision when mapping m6Am. We envision that it is also plausible to use m6ACE-seq to map PCIF1-dependent m6Am maps as an alternative to CAGE-seq for TSS mapping. Finally, our work also addresses an ongoing debate about whether the existence of m6A demethylases means that m6A and m6Am are reversible modifications (Darnell et al., 2018; Ke et al., 2017; Rosa-Mercado et al., 2017; Zhao et al., 2018). Such a model purports that after RNA accumulates functional levels of methylation via methyltransferases in the nucleus, it can undergo methylation-mediated regulation before being demethylated in the cytoplasm, allowing the newly-unmethylated RNA to again function in the absence of RNA methylation (FIG. 7g). Based on analysis of our methylome atlas, we instead propose the following (FIG. 7h): Most RNAs that are methylated do not undergo demethylation and remain methylated until they decay in the cytoplasm. However, methylation of selected RNA sites can disrupt downstream RNA processing pathways and thus, these RNA sites are supposed to remain unmethylated. Despite this, these selected RNA sites are still targeted by methylases, perhaps because they fulfill the consensus target motif. As such, demethylases actively and specifically demethylate these RNA sites while they are in the nucleus so as to suppress disruptive methylation from ever accumulating. Failure of demethylases to do this subjects these RNAs to unwanted regulatory pathways, which can have broad implications on cellular processes. We illustrate this using U4 snRNA, which has no quantifiable methylation in WT cells but accumulates m6Am at its first nucleotide in the absence of FTO. Perhaps due to steric hindrance by the cap-adjacent m6Am, binding of the U4 snRNA precursor to nuclear export machinery is reduced, potentially impeding the assembly of spliceosomes available for pre-mRNA splicing (Kiss, 2004). This likely contributes to the aberrant widespread exon exclusion phenotype previously observed in Fto-KO cells (Bartosovic et al., 2017). It is noteworthy that out of all Sm-class snRNAs, the 5′ terminal of U4 snRNA best fits the PCIF1-dependent ‘Cm6AmBU’ consensus motif. Therefore, U4 snRNA is likely to be the snRNA most targeted by PCIF1, which explains why loss of demethylation by FTO affects U4 snRNA more severely than other Sm-class snRNAs.

Our data do not discount the possibility that m6A and m6Am might be dynamically reversed in response to certain cellular stresses or developmental triggers that cause functional ALKBH5 or FTO to translocate into the cytoplasm.

Should such conditions ever be identified, use of m6ACE-seq will certainly help to validate any reversal of RNA methylation. Additionally, how demethylases are directed to suppress methylation from accumulating on specific RNA targets and if this specificity changes in different tissues remain to be determined. We envision that an atlas of single-base-resolution methylomes in different cell types will help elucidate demethylase specificity mechanisms and identify new forms of RNA-methylation-mediated RNA metabolism.

### REFERENCES

Any listing or discussion of an apparently prior-published document in this specification should not necessarily be taken as an acknowledgement that such document is part of the state of the art or is common general knowledge.
Aas, A., Isakson, P., Bindesbøll, C., Alemu, E. A., Klungland, A., Simonsen, A., 2017. Nucleocytoplasmic Shuttling of FTO Does Not Affect Starvation-Induced Autophagy. PLoS ONE 12, e0168182-15. doi:10.1371/journal.pone.0168182
Abugessaisa, I., Noguchi, S., Hasegawa, A., Harshbarger, J., Kondo, A., Lizio, M., Severin, J., Carninci, P., Kawaji, H., Kasukawa, T., 2017. FANTOM5 CAGE profiles of human and mouse reprocessed for GRCh38 and GRCm38 genome assemblies. Sci. Data 4, 170107-10. doi:10.1038/sdata.2017.107
Akichika, S., Hirano, S., Shichino, Y., Suzuki, T., Nishimasu, H., Ishitani, R., Sugita, A., Hirose, Y., Iwasaki, S., Nureki, O., Suzuki, T., 2018. Cap-specific terminal N6-methylation of RNA by an RNA polymerase II-associated methyltransferase. Science 44, eaav0080-13. doi:10.1126/science.aav0080
Alarcón, C. R., Goodarzi, H., Lee, H., Liu, X., Tavazoie, S., Tavazoie, S. F., 2015a. HNRNPA2B1 Is a Mediator of m6A-Dependent Nuclear RNA Processing Events. Cell 162, 1299-1308. doi:10.1016/j.cell.2015.08.011
Alarcon, C. R., Lee, H., Goodarzi, H., Halberg, N., Tavazoie, S. F., 2015b. N6-methyladenosine marks primary microRNAs for processing. Nature 519, 1-16. doi:10.1038/nature14281
Bartosovic, M., Molares, H. C., Gregorova, P., Hrossova, D., Kudla, G., Vanacova, S., 2017. N6-methyladenosine demethylase FTO targets pre-mRNAs and regulates alternative splicing and 3′-end processing. Nucleic Acids Res. 45, 11356-11370. doi:10.1093/nar/gkx778
Batista, P. J., Molinie, B., Wang, J., Qu, K., Zhang, J., Li, L., Bouley, D. M., Lujan, E., Haddad, B., Daneshvar, K., Carter, A. C., Flynn, R. A., Zhou, C., Lim, K.-S., Dedon, P., Wernig, M., Mullen, A. C., Xing, Y., Giallourakis, C. C., Chang, H. Y., 2014. m(6)A RNA modification controls cell fate transition in mammalian embryonic stem cells. Cell Stem Cell 15, 707-719. doi:10.1016/j.stem.2014.09.019
Bokar, J. A., Rath-Shambaugh, M. E., Ludwiczak, R., Narayan, P., Rottman, F., 1994. Characterization and partial purification of mRNA N6-adenosine methyltransferase from HeLa cell nuclei. Internal mRNA methylation requires a multisubunit complex. Journal of Biological Chemistry 269, 17697-17704.
Bokar, J. A., Shambaugh, M. E., Polayes, D., Matera, A. G., Rottman, F. M., 1997. Purification and cDNA cloning of the AdoMet-binding subunit of the human mRNA (N6-adenosine)-methyltransferase. RNA 3, 1233-1247.
Darnell, R. B., Ke, S., Darnell, J. E., 2018. Pre-mRNA processing includes N6methylation of adenosine residues that are retained in mRNA exons and the fallacy of “RNA epigenetics”. RNA 24, 262-267. doi:10.1261/rna.065219.117
Dobin, A., Davis, C. A., Schlesinger, F., Drenkow, J., Zaleski, C., Jha, S., Batut, P., Chaisson, M., Gingeras, T. R., 2013. STAR: ultrafast universal RNA-seq aligner. Bioinformatics 29, 15-21. doi:10.1093/bioinformatics/bts635
Dominissini, D., Moshitch-Moshkovitz, S., Schwartz, S., Salmon-Divon, M., Ungar, L., Osenberg, S., Cesarkas, K., Jacob-Hirsch, J., Amariglio, N., Kupiec, M., Sorek, R., Rechavi, G., 2012. Topology of the human and mouse m6A RNA methylomes revealed by m6A-seq. Nature 485, 201-206. doi:10.1038/nature11112
Geula, S., Moshitch-Moshkovitz, S., Dominissini, D., Mansour, A. A., Kol, N., Salmon-Divon, M., Hershkovitz, V., Peer, E., Mor, N., Manor, Y. S., Ben-Haim, M. S., Eyal, E., Yunger, S., Pinto, Y., Jaitin, D. A., Viukov, S., Rais, Y., Krupalnik, V., Chomsky, E., Zerbib, M., Maza, I., Rechavi, Y., Massarwa, R., Hanna, S., Amit, I., Levanon, E. Y., Amariglio, N., Stern-Ginossar, N., Novershtern, N., Rechavi, G., Hanna, J. H., 2015. m6A mRNA methylation facilitates resolution of naïve pluripotency toward differentiation. Science. doi:10.1126/science.1261417
Goh, W. S., Orlov, Y., Li, J., Clarke, N. D., 2010. Blurring of high-resolution data shows that the effect of intrinsic nucleosome occupancy on transcription factor binding is mostly regional, not local. PLoS Comput Biol 6, e1000649.
Goh, W. S. S., Falciatori, I., Tam, O. H., Burgess, R., Meikar, O., Kotaja, N., Hammell, M., Hannon, G. J., 2015. piRNA-directed cleavage of meiotic transcripts regulates spermatogenesis. Genes & Development 29, 1032-1044. doi:10.1101/gad.260455.115
Goh, W. S. S., Seah, J. W. E., Harrison, E. J., Chen, C., Hammell, C. M., Hannon, G. J., 2014. A genome-wide RNAi screen identifies factors required for distinct stages of C. elegans piRNA biogenesis. Genes & Development 28, 797-807. doi:10.1101/gad.235622.113
Iyer, L. M., Abhiman, S., Aravind, L., 2011. Natural history of eukaryotic DNA methylation systems. Prog Mol Biol Transl Sci 101, 25-104. doi:10.1016/B978-0-12-387685-0.00002-0
Jia, G., Fu, Y., Zhao, X., Dai, Q., Zheng, G., Yang, Y., Yi, C., Lindahl, T., Pan, T., Yang, Y.-G., 2011. N6-methyladenosine in nuclear RNA is a major substrate of the obesity-associated FTO. Nature Chemical Biology 7, 885-887. doi:10.1038/nchembio.687
Karijolich, J., Yu, Y.-T., 2010. Spliceosomal snRNA modifications and their function. RNA Biol 7, 192-204. doi:10.4161/rna.7.2.11207
Ke, S., Alemu, E. A., Mertens, C., Gantman, E. C., Fak, J. J., Mele, A., Haripal, B., Zucker-Scharff, I., Moore, M. J., Park, C. Y., Vågbø, C. B., Kusśnierczyk, A.,
Klungland, A., Darnell, J. E., Darnell, R. B., 2015. A majority of m6A residues are in the last exons, allowing the potential for 3′ UTR regulation. Genes & Development 29, 2037-2053. doi:10.1101/gad.269415.115
Ke, S., Pandya-Jones, A., Saito, Y., Fak, J. J., Vågbø, C. B., Geula, S., Hanna, J. H., Black, D. L., Darnell, J. E., Darnell, R. B., 2017. m6A mRNA modifications are deposited in nascent pre-mRNA and are not required for splicing but do specify cytoplasmic turnover. Genes & Development 31, 990-1006. doi:10.1101/gad.301036.117
Kiss, T., 2004. Biogenesis of small nuclear RNPs. Journal of Cell Science 117, 5949-5951. doi:10.1242/jcs.01487
Koh, C. W. Q., Goh, Y. T., Toh, J. D. W., Neo, S. P., Ng, S. B., Gunaratne, J., Gao, Y.-G., Quake, S. R., Burkholder, W. F., Goh, W. S. S., 2018. Single-nucleotide-resolution sequencing of human N6-methyldeoxyadenosine reveals strand-asymmetric clusters associated with SSBP1 on the mitochondrial genome. Nucleic Acids Res. 14, 204-12. doi:10.1093/nar/gky1104
Langmead, B., Salzberg, S. L., 2012. Fast gapped-read alignment with Bowtie 2. Nature Methods 9, 357-359. doi:10.1038/nmeth.1923
Linder, B., Grozhik, A. V., Olarerin-George, A. O., Meydan, C., Mason, C. E., Jaffrey, S. R., 2015. Single-nucleotide-resolution mapping of m6A and m6Am throughout the transcriptome. Nature Methods 12, 767-772. doi:10.1038/nmeth.3453
Liu, J., Yue, Y., Han, D., Wang, X., Fu, Y., Zhang, L., Jia, G., Yu, M., Lu, Z., Deng, X., Dai, Q., Chen, W., He, C., 2013. A METTL3-METTL14 complex mediates mammalian nuclear RNA N6-adenosine methylation. Nature Chemical Biology 10, 93-95. doi:10.1038/nchembio.1432
Liu, N., Parisien, M., Dai, Q., Zheng, G., He, C., Pan, T., 2013. Probing N6-methyladenosine RNA modification status at single nucleotide resolution in mRNA and long noncoding RNA. RNA 19, 1848-1856. doi:10.1261/rna.041178.113
Liu, W., Yan, J., Zhang, Z., Pian, H., Liu, C., Li, Z., 2018. Identification of a selective DNA ligase for accurate recognition and ultrasensitive quantification of N6-methyladenosine in RNA at one-nucleotide resolution. Chemical Science 9, 3354-3359. doi:10.1039/C7SC05233B
Love, M. I., Huber, W., Anders, S., 2014. Moderated estimation of fold change and dispersion for RNA-seq data with DESeq2. Genome Biol. 15, 31-21. doi:10.1186/s13059-014-0550-8
Machanick, P., Bailey, T. L., 2011. MEME-ChIP: motif analysis of large DNA datasets. Bioinformatics 27, 1696-1697. doi:10.1093/bioinformatics/btr189 Martin, M., 2011. Cutadapt removes adapter sequences from high-throughput sequencing reads. EMBnet. journal 17, 3-12. doi:10.14806/ej.17.1.200
Mauer, J., Luo, X., Blanjoie, A., Jiao, X., Grozhik, A. V., Patil, D. P., Linder, B., Pickering, B. F., Vasseur, J.-J., Chen, Q., Gross, S. S., Elemento, O., Debart, F., Kiledjian, M., Jaffrey, S. R., 2017. Reversible methylation of m6Am in the 5′ cap controls mRNA stability. Nature 541, 371-375. doi:10.1038/nature21022
Mauer, J., Sindelar, M., Despic, V., Guez, T., Hawley, B. R., Vasseur, J.-J., Rentmeister, A., Gross, S. S., Pellizzoni, L., Debart, F., Goodarzi, H., Jaffrey, S. R., 2019. FTO controls reversible m6Am RNA methylation during snRNA biogenesis. Nature Chemical Biology 15, 340-347. doi:10.1038/s41589-019-0231-8
Meyer, K. D., Patil, D. P., Zhou, J., Zinoviev, A., Skabkin, M. A., 2015. 5′ UTR m 6 A Promotes Cap-Independent Translation. Cell 163, 999-1010. doi:10.1016/j.cell.2015.10.012
Meyer, K. D., Saletore, Y., Zumbo, P., Elemento, O., Mason, C. E., Jaffrey, S. R., 2012. Comprehensive analysis of mRNA methylation reveals enrichment in 3′ UTRs and near stop codons. Cell 149, 1635-1646. doi:10.1016/j.cell.2012.05.003
Mi, H., Huang, X., Muruganujan, A., Tang, H., Mills, C., Kang, D., Thomas, P. D., 2017. PANTHER version 11: expanded annotation data from Gene Ontology and Reactome pathways, and data analysis tool enhancements. Nucleic Acids Res. 45, D183-D189. doi:10.1093/nar/gkw1138
Moss, B., GERSHOWITZ, A., Weber, L. A., Baglioni, C., 1977. Histone mRNAs contain blocked and methylated 5′ terminal sequences but lack methylated nucleosides at internal positions. Cell 10, 113-120. doi:10.1016/0092-8674(77)90145-3
Olarerin-George, A. O., Jaffrey, S. R., 2017. MetaPlotR: a Perl/R pipeline for plotting metagenes of nucleotide modifications and other transcriptomic sites. Bioinformatics 33, 1563-1564. doi:10.1093/bioinformatics/btx002
Patil, D. P., Chen, C.-K., Pickering, B. F., Chow, A., Jackson, C., Guttman, M., Jaffrey, S. R., 2016. m6A RNA methylation promotes XIST-mediated transcriptional repression. Nature 537, 369-373. doi:10.1038/nature19342
Pendleton, K. E., Chen, B., Liu, K., Hunter, O. V., Xie, Y., Tu, B. P., Conrad, N. K., 2017. The U6 snRNA m6A Methyltransferase METTL16 Regulates SAM Synthetase Intron Retention. Cell 169, 824-829.e14. doi:10.1016/j.cell.2017.05.003
Piekna-Przybylska, D., Decatur, W. A., Fournier, M. J., 2008. The 3D rRNA modification maps database: with interactive tools for ribosome analysis. Nucleic Acids Res. 36, D178-D183. doi:10.1093/nar/gkm855
Ping, X.-L., Sun, B.-F., Wang, L., Xiao, W., Yang, X., Wang, W.-J., Adhikari, S., Shi, Y., Lv, Y., Chen, Y.-S., 2014. Mammalian WTAP is a regulatory subunit of the RNA N6-methyladenosine methyltransferase. Cell Res. 24, 177-189. doi:10.1038/cr.2014.3
Rosa-Mercado, N. A., Withers, J. B., Steitz, J. A., 2017. Settling the m6A debate: methylation of mature mRNA is not dynamic but accelerates turnover. Genes & Development 31, 957-958. doi:10.1101/gad.302695.117
Schwartz, S., Mumbach, M. R., Jovanovic, M., Wang, T., Maciag, K., Bushkin, G. G., Mertins, P., Ter-Ovanesyan, D., Habib, N., Cacchiarelli, D., Sanjana, N. E., Freinkman, E., Pacold, M. E., Satija, R., Mikkelsen, T. S., Hacohen, N., Zhang, F., Carr, S. A., Lander, E. S., Regev, A., 2014. Perturbation of m6A writers reveals two distinct classes of mRNA methylation at internal and 5′ sites. CellReports 8, 284-296. doi:10.1016/j.celrep.2014.05.048
Sun, H., Zhang, M., Li, K., Bai, D., Yi, C., 2018. Cap-specific, terminal N6-methylation by a mammalian m6Am methyltransferase. Cell Res. 169, 1-3. doi:10.1038/s41422-018-0117-4
Vo ngoc, L., Cassidy, C. J., Huang, C. Y., Duttke, S. H. C., Kadonaga, J. T., 2017. The human initiator is a distinct and abundant element that is precisely positioned in focused core promoters. Genes & Development 31, 6-11. doi:10.1101/gad.293837.116
Wang, Xiang, Feng, J., Xue, Y., Guan, Z., Zhang, D., Liu, Z., Gong, Z., Wang, Q., Huang, J., Tang, C., Zou, T., Yin, P., 2016. Structural basis of N6-adenosine methylation by the METTL3-METTL14 complex. Nature 534, 575-578. doi:10.1038/nature18298
Wang, Xiao, Lu, Z., Gomez, A., Hon, G. C., Yue, Y., Han, D., Fu, Y., Parisien, M., Dai, Q., Jia, G., Ren, B., Pan, T., He, C., 2014. N6-methyladenosine-dependent regulation of messenger RNA stability. Nature 505, 117-120. doi:10.1038/nature12730
Wang, Xiao, Zhao, B. S., Roundtree, I. A., Lu, Z., Han, D., Ma, H., Weng, X., Chen, K., Shi, H., He, C., 2015. N(6)-methyladenosine Modulates Messenger RNA Translation Efficiency. Cell 161, 1388-1399. doi:10.1016/j.cell.2015.05.014
Wei, C., Gershowitz, A., Moss, B., 1975. N6,O2″-dimethyladenosine a novel methylated ribonucleoside next to the 5″ terminal of animal cell and virus mRNAs. Nature 257, 251-253.
Wei, J., Liu, F., Lu, Z., Fei, Q., Ai, Y., He, P. C., Shi, H., Cui, X., Su, R., Klungland, A., Jia, G., Chen, J., He, C., 2018. Differential m6A, m6Am, and m1A Demethylation Mediated by FTO in the Cell Nucleus and Cytoplasm. Molecular Cell 1-19. doi:10.1016/j.molcel.2018.08.011
Zhao, B. S., Nachtergaele, S., Roundtree, I. A., He, C., 2018. Our views of dynamic N6-methyladenosine RNA methylation. RNA 24, 268-272. doi:10.1261/rna.064295.117
Zheng, G., Dahl, J. A., Niu, Y., Fedorcsak, P., Huang, C.-M., Li, C. J., VågbØ, C. B., Shi, Y., Wang, W.-L., Song, S.-H., Lu, Z., Bosmans, R. P. G., Dai, Q., Hao, Y.-J., Yang, X., Zhao, W.-M., Tong, W.-M., Wang, X.-J., Bogdan, F., Furu, K., Fu, Y., Jia, G., Zhao, X., Liu, J., Krokan, H. E., Klungland, A., Yang, Y.-G., He, C., 2013. ALKBH5 is a mammalian RNA demethylase that impacts RNA metabolism and mouse fertility. Molecular Cell 49, 18-29. doi:10.1016/j.molcel.2012.10.015

1. A method for analysis of methylation of ribonucleic acid (RNA) comprising the steps:
(i) contacting RNA with one or more antibodies which binds to methylated site(s) of RNA; wherein the methylated site(s) comprise at least one ribonucleotide base modified by one or more methyl groups;
(ii) photo-crosslinking the one or more antibodies to crosslink individual antibodies to the RNA molecule(s) to form RNA-antibody conjugates;
(iii) immunoprecipitating to separate the RNA-antibody conjugates;
(iv) treating the RNA-antibody conjugates with at least one exonuclease;
(v) removing the crosslinked antibodies from the RNA-antibody conjugates to release RNA; and
(vi) analysing the released RNA.

2. The method according to claim 1, wherein the method further comprises ligating first adapter nucleic acid molecules to the 3′ end of the RNA molecule(s).

3. The method according to claim 2, further comprising ligating second adapter nucleic acid molecules to the 5′ end of the RNA molecule(s) after treatment with exonuclease.

4. The method according to claim 1, wherein the exonuclease comprises a 5′ to 3′ exonuclease.

5. The method according to claim 1, wherein analysing the RNA comprises reverse transcribing the released RNA to complementary deoxyribonucleic acid (cDNA) and analysing the cDNA.

6. The method according to any one of claims 2 to 5, comprising reverse transcribing the released RNA using an oligonucleotide molecule substantially complementary to the first adaptor molecule to form single stranded complementary deoxyribonucleic acid (cDNA).

7. The method according to claim 6, further comprising amplifying the single stranded cDNA by polymerase chain reaction (PCR) to form double stranded cDNA (ds cDNA).

8. The method according to claim 7, comprising using a first primer substantially complementary to the first adaptor molecule and a second primer substantially complementary to the second adapter molecule for the PCR amplification.

9. The method according to claim 7, further comprising analysing the ds cDNA.

10. The method according to claim 5 or 9, wherein analysing the cDNA or ds cDNA comprises sequencing and/or mapping the cDNA or ds cDNA.


SEQUENCE LISTING (35 sequences)

SEQ ID NO: 1 - RNA, 57 nt, Artificial Sequence
  Methylated RNA spike-in
  n (21)..(21): N6-methyladenosine
  misc_feature (21)..(21): n is a, c, g, or u
ucuccuuaguagcuccuaagnucgucgaguuacacgacgacauuguuccgacugacg

SEQ ID NO: 2 - DNA, 20 bp, artificial
  Mettl3 sgRNA #1
gagagtccagctgcttcttg

SEQ ID NO: 3 - DNA, 20 bp, artificial
  Mettl3 sgRNA #2
ctatatcctggagcgagtgc

SEQ ID NO: 4 - DNA, 20 bp, artificial
  Pcif1 sgRNA #1
gcagggacgcttcctcccgg

SEQ ID NO: 5 - DNA, 20 bp, artificial
  Pcif1 sgRNA #2
aggtcctgaaccaggcggat

SEQ ID NO: 6 - DNA, 20 bp, artificial
  Alkbh5 sgRNA #1
cctcatagtcgctgcgctcg

SEQ ID NO: 7 - DNA, 20 bp, artificial
  Alkbh5 sgRNA #2
atagttgtcccgggacgtca

SEQ ID NO: 8 - DNA, 20 bp, artificial
  Fto sgRNA #1
ctaaatcccgtggcgctcgc

SEQ ID NO: 9 - DNA, 19 bp, artificial
  Fto sgRNA #2
agcttcgcgctctcgttcc

SEQ ID NO: 10 - DNA, 22 bp, artificial
  3′ Ligation DNA Adapter
  n (1)..(1): 5′ adenylation
  misc_feature (1)..(1): n is a, c, g, or t
  n (22)..(22): dideoxycytosine
  misc_feature (22)..(22): n is a, c, g, or t
nggaattctcgggtgccaaggn

SEQ ID NO: 11 - RNA, 34 nt, artificial
  5′ Ligation RNA Adapter
  n (17)..(33): n can be A, U, C or G
  misc_feature (27)..(34): n is a, c, g, or u
  n (34)..(34): n can be C, G or U
guucagaguucuacaguccgacgaucnnnnnnnn

SEQ ID NO: 12 - DNA, 22 bp, artificial
  m6ACe Reverse Transcription Primer
gccttggcacccgagaattcca

SEQ ID NO: 13 - DNA, 21 bp, artificial
  qPCR 7SL scRNA for
atcgggtgtccgcactaagtt

SEQ ID NO: 14 - DNA, 20 bp, artificial
  qPCR 7SL scRNA rev
cagcacgggagttttgacct

SEQ ID NO: 15 - DNA, 19 bp, artificial
  qPCR 5.8S rRNA for
ggtggatcactcggctcgt

SEQ ID NO: 16 - DNA, 20 bp, artificial
  qPCR 5.8S rRNA rev
gcaagtgcgttcgaagtgtc

SEQ ID NO: 17 - DNA, 21 bp, artificial
  qPCR U1 snRNA for
ccatgatcacgaaggtggttt

SEQ ID NO: 18 - DNA, 21 bp, artificial
  qPCR U1 snRNA rev
atgcagtcgagtttcccacat

SEQ ID NO: 19 - DNA, 20 bp, artificial
  qPCR U2 snRNA for
ttctcggccttttggctaag

SEQ ID NO: 20 - DNA, 20 bp, artificial
  qPCR U2 snRNA rev
ctccctgctccaaaaatcca

SEQ ID NO: 21 - DNA, 21 bp, artificial
  qPCR U4 snRNA for
gccaatgaggtttatccgagg

SEQ ID NO: 22 - DNA, 21 bp, artificial
  qPCR U4 snRNA rev
caaaaattgccaatgccgact

SEQ ID NO: 23 - DNA, 24 bp, artificial
  qPCR U5 snRNA for
tggtttctcttcagatcgcataaa

SEQ ID NO: 24 - DNA, 20 bp, artificial
  qPCR U5 snRNA rev
gccaaagcaaggcctcaaaa

SEQ ID NO: 25 - DNA, 22 bp, artificial
  qPCR Gapdh for
gtctcctctgacttcaacagcg

SEQ ID NO: 26 - DNA, 22 bp, artificial
  qPCR Gapdh rev
accaccctgttgctgtagccaa

SEQ ID NO: 27 - DNA, 34 bp, artificial
  Novel m6A T3LIG probe L; 5′ phosphorylated
aacagttaaatttactctatgggcagtcggtgat

SEQ ID NO: 28 - DNA, 33 bp, artificial
  Novel m6A T3LIG probe R
  n (31)..(31): RNA version of A
  misc_feature (31)..(32): n is a, c, g, t or u
  n (32)..(32): RNA version of C
ccatctcatccctgcgtgtcccctctttggnnu

SEQ ID NO: 29 - DNA, 34 bp, artificial
  Non-m6A 1 T3LIG probe L; 5′ phosphorylated
agtgtccaaagagcttctatgggcagtcggtgat

SEQ ID NO: 30 - DNA, 33 bp, artificial
  Non-m6A 1 T3LIG probe R
  n (31)..(31): RNA version of C
  misc_feature (31)..(32): n is a, c, g, t or u
  n (32)..(32): RNA version of C
ccatctcatccctgcgtgtccaggttttttnnu

SEQ ID NO: 31 - DNA, 34 bp, artificial
  Non-m6A 2 T3LIG probe L; 5′ phosphorylated
actatgggtgttaaatctatgggcagtcggtgat

SEQ ID NO: 32 - DNA, 33 bp, artificial
  Non-m6A 2 T3LIG probe R
  n (31)..(31): RNA version of C
  misc_feature (31)..(32): n is a, c, g, t or u
  n (32)..(32): RNA version of C
ccatctcatccctgcgtgtccgcttttaggnnu

SEQ ID NO: 33 - DNA, 19 bp, artificial
  Universal qPCR primer for T3 DNA ligase assay for
atcaccgactgcccataga

SEQ ID NO: 34 - DNA, 20 bp, artificial
  Universal qPCR primer for T3 DNA ligase assay rev
ccatctcatccctgcgtgtc

SEQ ID NO: 35 - RNA, 20 nt, artificial
  Methylated RNA for in vitro demethylation
  n (10)..(10): N6-methyladenosine
  misc_feature (10)..(10): n is a, c, g, or u
ccgucaaggncugucucugc